(19) United States
(12) Patent Application Publication
(10) Pub. No.: US 2026/0007441 A1
(43) Pub. Date: Jan. 8, 2026

(54) SOFT TISSUE RECONSTRUCTION DEVICES, SYSTEMS, AND METHODS

(71) Applicant: RTG Scientific, LLC, Austin, TX (US)

(72) Inventors: Mary SINNOTT, Logan, UT (US); Andrew FAUTH, North Logan, UT (US)

(21) Appl. No.: 19/252,910

(22) Filed: Jun. 27, 2025

Related U.S. Application Data
(60) Provisional application No. 63/667,891, filed on Jul. 5, 2024.

(51) Int. Cl. A61B 17/86; A61B 17/00; A61B 17/68

(52) U.S. Cl. CPC A61B 17/8645; A61B 17/685; A61B 2017/00477; A61B 2017/8655

(57) ABSTRACT

A system for reconstructing a soft tissue may include a first interference bone screw having a first shaft and a first helical thread with a first concave undercut surface, and a second interference bone screw having a second shaft and a second helical thread with a second concave undercut surface. The first helical thread of the first interference bone screw may be configured to couple a first portion of a soft tissue implant within a first bone tunnel formed in a first bone, and the second helical thread of the second interference bone screw may be configured to couple a second portion of the soft tissue implant within a second bone tunnel formed in a second bone.

### CROSS-REFERENCE TO RELATED APPLICATIONS

This application claims the benefit of U.S. Provisional Patent Application Ser. No. 63/667,891 filed on Jul. 5, 2024, entitled “SOFT TISSUE RECONSTRUCTION DEVICES, SYSTEMS, AND METHODS”. The above-referenced document is hereby incorporated by reference in its entirety.

### TECHNICAL FIELD

The present disclosure relates to medical devices, systems, and methods. More specifically, the present disclosure relates to soft tissue reconstruction devices, systems, and methods with improved thread designs for coupling soft tissue implants to bone.

### BACKGROUND

Interference bone screws have been utilized in various soft tissue reconstruction scenarios, such as Anterior Cruciate Ligament (ACL) reconstruction procedures, etc.

However, the interference bone screw designs typically utilized in such procedures depend on outward radial forces to hold the reconstructed ACL implant within the bone tunnels that are formed in the femur and tibia, and do not fully engage/grip the walls of the bone tunnels and/or sufficiently retain the reconstructed ACL implant placed therein.

Accordingly, improved interference bone screw designs that can fully engage/grip the walls of the bone tunnels and/or increase fixation of the reconstructed ACL implant that is placed therein would be desirable.

### SUMMARY

The interference bone screw devices, systems, and methods of the present disclosure have been developed in response to the present state of the art, and in particular, in response to the problems and needs in the art that have not yet been fully solved by currently available interference bone screw devices, systems, and methods. In some embodiments, the interference bone screw devices, systems, and methods of the present disclosure may provide improved devices, systems, and methods for coupling soft tissue implants to bone.

In some embodiments, a system for reconstructing a soft tissue may include a first interference bone screw and a second interference bone screw. The first interference bone screw may include a first shaft having a first proximal end, a first distal end, and a first longitudinal axis. The first interference bone screw may also include a first helical thread disposed about the first shaft along the first longitudinal axis between a first location and a second location along the first shaft, the first helical thread having a first concave undercut surface. The second interference bone screw may include a second shaft having a second proximal end, a second distal end, and a second longitudinal axis. The second interference bone screw may also include a second helical thread disposed about the second shaft along the second longitudinal axis between a third location and a fourth location along the second shaft, the second helical thread having a second concave undercut surface. The first helical thread of the first interference bone screw may be configured to interlock with of a first bone block, and the first bone block may be placed intermediate the first interference bone screw and a first portion of a soft tissue implant within a first bone tunnel formed in a first bone. The second helical thread of the second interference bone screw may be configured to interlock with a second bone block, and the second bone block may be placed intermediate the second interference bone screw and a second portion of the soft tissue implant to couple the second portion of the soft tissue implant within a second bone tunnel formed in a second bone.

The system according to any preceding paragraph, wherein the first concave undercut surface of the first helical thread may include a standard thread orientation, and the second concave undercut surface of the second helical thread may include an inverted thread orientation.

The system according to any preceding paragraph, wherein the first concave undercut surface of the first helical thread may include an inverted thread orientation, and the second concave undercut surface of the second helical thread may include a standard thread orientation.

The system according to any preceding paragraph, wherein the first concave undercut surface of the first helical thread may include a first standard thread orientation, and the second concave undercut surface of the second helical thread may include a second standard thread orientation.

The system according to any preceding paragraph, wherein the first concave undercut surface of the first helical thread may include a first inverted thread orientation, and the second concave undercut surface of the second helical thread may include a second inverted thread orientation.

The system according to any preceding paragraph, wherein at least one of the first bone block may include a first pre-formed thread configured to receive the first helical thread therein, and the second bone block may include a second pre-formed thread configured to receive the second helical thread therein.

The system according to any preceding paragraph, wherein at least one of the first bone tunnel may include a first bone tunnel pre-formed thread configured to receive the first helical thread therein, and the second bone tunnel may include a second bone tunnel pre-formed thread configured to receive the second helical thread therein.

In some embodiments, a system for reconstructing a soft tissue may include a first interference bone screw and a second interference bone screw. The first interference bone screw may include a first shaft having a first proximal end, a first distal end, and a first longitudinal axis, as well as a first helical thread disposed about the first shaft along the first longitudinal axis between a first location and a second location along the first shaft, the first helical thread having a first concave undercut surface. The second interference bone screw may include a second shaft having a second proximal end, a second distal end, and a second longitudinal axis, as well as a second helical thread disposed about the second shaft along the second longitudinal axis between a third location and a fourth location along the second shaft, the second helical thread having a second concave undercut surface. One of the first interference bone screw and the second interference bone screw may be configured to interlock with a bone block engaged with a first portion of a soft tissue implant to couple the first portion of the soft tissue implant within a first bone tunnel formed in a first bone. The other one of the first interference bone screw and the second interference bone screw may be configured to interlock with a second portion of the soft tissue implant to couple the second portion of the soft tissue implant within a second bone tunnel formed in a second bone.

The system according to any preceding paragraph, wherein the first concave undercut surface may include at least one of a first standard thread orientation and a first inverted thread orientation, and the second concave undercut surface may include at least one of a second standard thread orientation and a second inverted thread orientation.

The system according to any preceding paragraph, wherein the first interference bone screw may include at least one first minor diameter, and the second interference bone screw may include at least one second minor diameter.

The system according to any preceding paragraph, wherein the at least one first minor diameter may be constant along at least a portion of a first length of the first shaft, and the at least one second minor diameter may be constant along at least a portion of a second length of the second shaft.

The system according to any preceding paragraph, wherein the at least one first minor diameter may be configured to decrease in size toward the first distal end of the first shaft, and the at least one second minor diameter may be configured to decrease in size toward the second distal end of the second shaft.

The system according to any preceding paragraph, wherein the at least one first minor diameter may be configured to continuously decrease in size toward the first distal end of the first shaft, and the at least one second minor diameter may be configured to continuously decrease in size toward the second distal end of the second shaft.

The system according to any preceding paragraph, wherein the at least one first minor diameter may be configured to discretely decrease in size toward the first distal end of the first shaft, and the at least one second minor diameter may be configured to discretely decrease in size toward the second distal end of the second shaft.

In some embodiments, a system for reconstructing a soft tissue may include a first interference bone screw and a second interference bone screw. The first interference bone screw may include a first shaft having a first proximal end, a first distal end, and a first longitudinal axis, as well as a first helical thread disposed about the first shaft along the first longitudinal axis between a first location and a second location along the first shaft, the first helical thread having a first concave undercut surface. The second interference bone screw may include a second shaft having a second proximal end, a second distal end, and a second longitudinal axis, as well as a second helical thread disposed about the second shaft along the second longitudinal axis between a third location and a fourth location along the second shaft, the second helical thread having a second concave undercut surface. The first helical thread of the first interference bone screw may be configured to interlock with a first portion of a soft tissue implant to couple the first portion of the soft tissue implant within a first bone tunnel formed in a first bone. The second helical thread of the second interference bone screw may be configured to interlock with a second portion of the soft tissue implant to couple the second portion of the soft tissue implant within a second bone tunnel formed in a second bone.

The system according to any preceding paragraph, wherein the first concave undercut surface may include at least one of a first standard thread orientation and a first inverted thread orientation, and the second concave undercut surface comprises at least one of a second standard thread orientation and a second inverted thread orientation.

The system according to any preceding paragraph, wherein the first interference bone screw may include at least one first major diameter, and the second interference bone screw may include at least one second major diameter.

The system according to any preceding paragraph, wherein the at least one first major diameter may be constant along at least a portion of the first helical thread, and the at least one second major diameter may be constant along at least a portion of the second helical thread.

The system according to any preceding paragraph, wherein the at least one first major diameter may be configured to continuously decrease in size toward the first distal end of the first shaft, and the at least one second major diameter may be configured to continuously decrease in size toward the second distal end of the second shaft.

The system according to any preceding paragraph, wherein the at least one first major diameter may be configured to discretely decrease in size toward the first distal end of the first shaft, and the at least one second major diameter may be configured to discretely decrease in size toward the second distal end of the second shaft.

These and other features and advantages of the present disclosure will become more fully apparent from the following description and appended claims or may be learned by the practice of the interference bone screw devices, systems, and methods set forth hereinafter.

### BRIEF DESCRIPTION OF THE DRAWINGS

Exemplary embodiments of the present disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1A illustrates a front perspective view of a fastener, according to an embodiment of the present disclosure;

FIG. 1B illustrates a rear perspective view of the fastener of FIG. 1A;

FIG. 1C illustrates a side view of the fastener of FIG. 1A;

FIG. 1D illustrates a cross-sectional side view of the fastener of FIG. 1A taken along the line A-A in FIG. 1C;

FIG. 2 illustrates a partial cross-sectional side view of a fastener comprising crescent-shaped threading;

FIG. 3A illustrates a front perspective view of a fastener, according to another embodiment of the present disclosure;

FIG. 3B illustrates a rear perspective view of the fastener of FIG. 3A;

FIG. 3C illustrates a side view of the fastener of FIG. 3A;

FIG. 3D illustrates a cross-sectional side view of the fastener of FIG. 3A taken along the line B-B in FIG. 3C;

FIG. 4A illustrates a front perspective view of a fastener, according to another embodiment of the present disclosure;

FIG. 4B illustrates a rear perspective view of the fastener of FIG. 4A;

FIG. 4C illustrates a side view of the fastener of FIG. 4A;

FIG. 4D illustrates a cross-sectional side view of the fastener of FIG. 4A taken along the line C-C in FIG. 4C;

FIG. 5A illustrates a front perspective view of a fastener, according to another embodiment of the present disclosure;

FIG. 5B illustrates a rear perspective view of the fastener of FIG. 5A;

FIG. 5C illustrates a side view of the fastener of FIG. 5A;

FIG. 5D illustrates a cross-sectional side view of the fastener of FIG. 5A taken along the line D-D in FIG. 5C;

FIG. 6A illustrates a front perspective view of a fastener, according to another embodiment of the present disclosure;

FIG. 6B illustrates a rear perspective view of the fastener of FIG. 6A;

FIG. 6C illustrates a side view of the fastener of FIG. 6A;

FIG. 6D illustrates a cross-sectional side view of the fastener of FIG. 6A taken along the line E-E in FIG. 6C;

FIG. 7A illustrates a front perspective view of a fastener, according to another embodiment of the present disclosure;

FIG. 7B illustrates a rear perspective view of the fastener of FIG. 7A;

FIG. 7C illustrates a side view of the fastener of FIG. 7A;

FIG. 7D illustrates a cross-sectional side view of the fastener of FIG. 7A taken along the line F-F in FIG. 7C;

FIG. 8A illustrates a front perspective view of a fastener, according to another embodiment of the present disclosure;

FIG. 8B illustrates a rear perspective view of the fastener of FIG. 8A;

FIG. 8C illustrates a side view of the fastener of FIG. 8A;

FIG. 8D illustrates a cross-sectional side view of the fastener of FIG. 8A taken along the line G-G in FIG. 8C;

FIG. 9A illustrates a perspective view of a system for reconstructing a soft tissue in a bone, according to an embodiment of the present disclosure;

FIG. 9B illustrates a cross-sectional side view of the system of FIG. 9A;

FIG. 10A illustrates a perspective view of a bone block and soft tissue implant, according to an embodiment of the present disclosure;

FIG. 10B illustrates a side view of the bone block and soft tissue implant of FIG. 10A;

FIG. 11A illustrates a side view of a system for reconstructing an ACL in a knee joint, according to an embodiment of the present disclosure;

FIG. 11B illustrates a side view of the system of FIG. 11A in greater detail;

FIG. 12A illustrates a perspective top view of a system for reconstructing a soft tissue between a first bone and a second bone, according to an embodiment of the present disclosure;

FIG. 12B illustrates a cross-sectional side view of the system of FIG. 12A;

FIG. 13A illustrates a perspective top view of a system for reconstructing a soft tissue between a first bone and a second bone, according to another embodiment of the present disclosure;

FIG. 13B illustrates a cross-sectional side view of the system of FIG. 13A;

FIG. 14A illustrates a perspective top view of a system for reconstructing a soft tissue between a first bone and a second bone, according to another embodiment of the present disclosure;

FIG. 14B illustrates a cross-sectional side view of the system of FIG. 14A;

FIG. 15A illustrates a perspective top view of a system for reconstructing a soft tissue between a first bone and a second bone, according to another embodiment of the present disclosure; and

FIG. 15B illustrates a cross-sectional side view of the system of FIG. 15A.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

Although the present disclosure illustrates soft tissue reconstruction devices, systems, and methods with reference to an ACL reconstruction for a knee joint, it will be understood that the devices, systems, and methods disclosed herein may be utilized for any type of soft tissue reconstruction application anywhere in the body.

Any implant or implant system disclosed or contemplated herein may be implanted in bone via any surgical technique, including, but not limited to: open surgical techniques, minimally-invasive surgical techniques (e.g., implants inserted arthroscopically via small incisions, robot-assisted surgical techniques, etc.), without departing from the spirit or scope of the present disclosure.

As used herein, the term “soft tissue” or “soft tissue implant” can comprise any tissues or material(s) including, but not limited to: any autograft tissues, any allograft tissues, any xenograft tissues, any artificial or synthetic tissues/material(s), etc., or any combinations thereof. For example, any implant or implant system disclosed or contemplated herein may utilize any material or combination of materials, including, but not limited to: any plastics or polymers (e.g., polyethylene [PE], poly-ether-ether-ketone [PEEK], poly-lactic acid [PLA], poly-lactic-co-glycolic acid [PLGA], poly-glycolic acid [PGA], poly-methyl-methacrylate [PMMA], silicone, etc.), any metals or metal alloys (e.g., titanium, stainless steel, nitinol or cobalt chrome, etc.), any ceramics (e.g., alumina, zirconia, etc.), any bio-composites (e.g., tricalcium phosphate [TCP], hydroxyapatite, calcium carbonate, etc.), any biological materials (e.g., bone grafts, collagen, bone growth stimulation proteins/chemicals, etc.), any resorbable or non-resorbable materials, etc., without departing from the spirit or scope of the present disclosure.

It will be understood that any fastener, bone implant, interference bone screw, etc., described or contemplated herein may (or may not) include any thread configuration, feature, or morphology that is described or contemplated herein to achieve optimal fixation within a given bone or for a given soft tissue reconstruction scenario. Moreover, it will also be understood that any fastener, bone implant, interference bone screw, etc., described or contemplated herein may be utilized in conjunction with (or within) any device, system, method, procedure, or instrument that is described or contemplated herein to form any number of different combinations of devices, systems, methods, procedures, etc. Moreover, any of the devices, systems, and instruments described or contemplated herein may be combined in any manner to produce any number of different surgical kits.

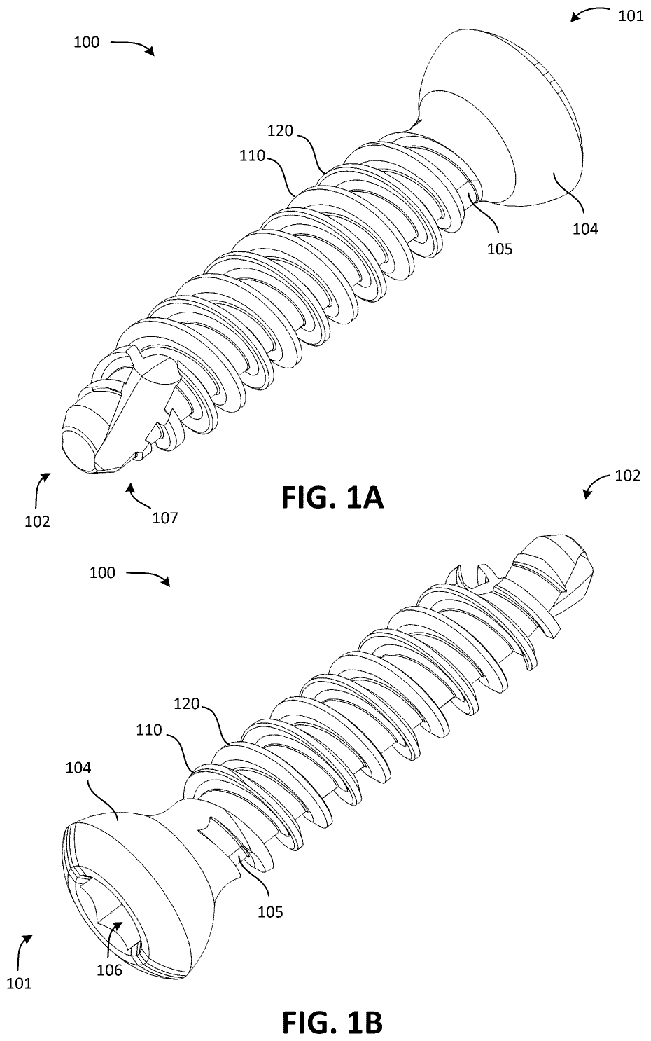

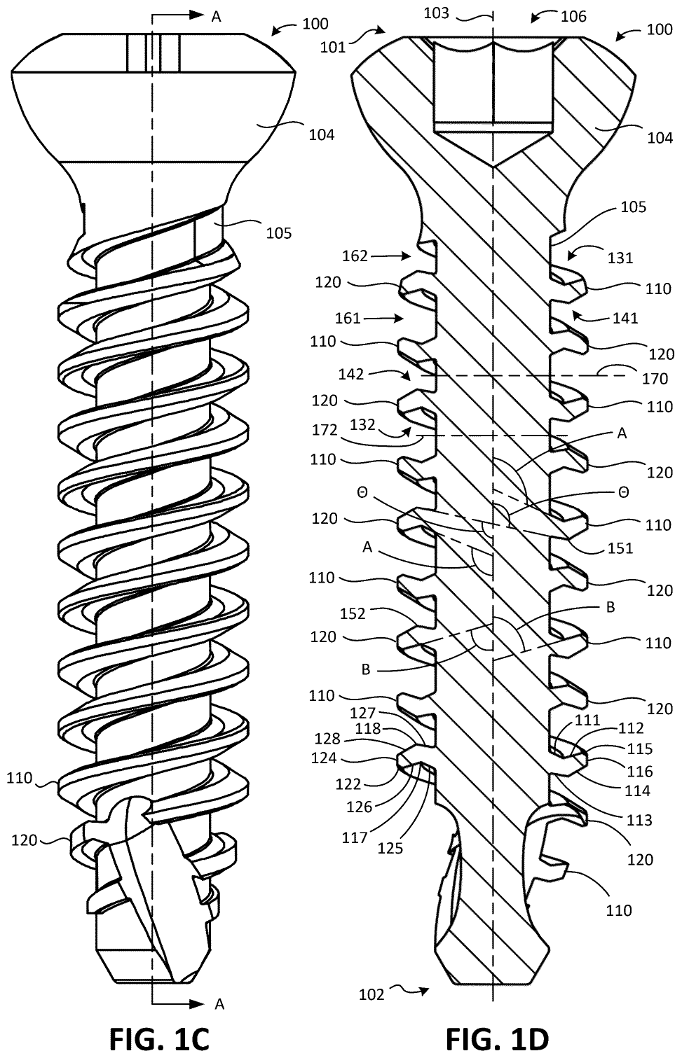

FIGS. 1A-1D illustrate various views of a device, screw, bone screw, bone implant, joint arthroplasty implant, interference bone screw, or fastener 100, according to an example of the present disclosure. Specifically, FIG. 1A is a perspective distal end view of the fastener 100, FIG. 1B is a perspective proximal end view of the fastener, FIG. 1C is a side view of the fastener, and FIG. 1D is a cross-sectional side view of the fastener taken along the line A-A in FIG. 1C.

In general, the fastener may include a shaft 105 having a proximal end 101, a distal end 102, and a longitudinal axis 103. The fastener may also include a head 104 located at the proximal end 101 of the shaft 105, a torque connection interface 106 formed in/on the head 104 (in either a male or female configuration), and a self-tapping feature 107 formed in the shaft 105, such as the distal end 102 of the shaft 105, etc.

In some embodiments, the fastener may include a first helical thread 110 disposed about the shaft 105, and/or a second helical thread 120 disposed about the shaft 105 adjacent the first helical thread 110.

In some embodiments, the fastener may include a “dual start” or “dual lead” thread configuration comprising the first helical thread 110 and the second helical thread 120.

In some embodiments, a depth of the first helical thread 110 and/or the second helical thread 120 with respect to the shaft 105 may define a major diameter vs. a minor diameter of the shaft 105 alone.

In some embodiments, a major diameter and/or a minor diameter of the fastener may be constant or substantially constant along the entire length of the fastener, along a majority of the length of the fastener, or along any length of the fastener. In these embodiments, a constant minor diameter may help avoid blowout of narrow/delicate bones (e.g., a pedicle or other bones) when inserting a fastener into a bone. In some embodiments, a pilot hole may first be drilled into a narrow/delicate bone and then a fastener having a similar minor diameter in comparison to the diameter of the pilot hole may be chosen to avoid blowout when inserting the fastener into the bone.

In some embodiments, a depth of the first helical thread 110 and/or the second helical thread 120 with respect to the shaft 105 may vary along a length of the shaft 105 to define one or more major diameters of the fastener, and/or one or more regions along the fastener may comprise one or more continuously variable major diameters.

In some embodiments, a thickness of the shaft 105 may vary along a length of the shaft 105 to define one or more minor diameters of the fastener, and/or one or more regions along the fastener may comprise one or more continuously variable minor diameters.

In some embodiments, a thickness/height/width/length/pitch/angle/shape, etc., of the first helical thread 110 and/or the second helical thread 120 (or any additional helical thread) may vary along a length of the shaft 105. For example, a thickness/height/width/length/pitch/angle/shape, etc., of the first helical thread 110 and/or the second helical thread 120 may be greater towards the tip of the fastener and thinner towards the head of the fastener (or vice versa) in either a discrete or continuously variable fashion, etc., or combinations thereof.

In some embodiments, the major and/or minor diameters may increase toward a proximal end or head of a fastener (or vice versa) in order to increase bone compaction as the fastener is terminally inserted into the bone/tissue.

In some embodiments, a pitch of the first helical thread 110 and/or the second helical thread 120 may vary along a length of the fastener.

In some embodiments, the fastener may include a plurality of helical threads disposed about the shaft 105. However, it will also be understood that any of the fasteners disclosed or contemplated herein may include a single helical thread disposed about the shaft of the fastener. Moreover, the fastener may comprise a nested plurality of helical threads having different lengths (not shown). As one non-limiting example, the fastener may include a first helical thread 110 that is longer than a second helical thread 120, such that the fastener comprises dual threading along a first portion of the shaft 105 and single threading along a second portion of the shaft 105.

In some embodiments, the plurality of helical threads may include three helical threads comprising a “triple start” or “triple lead” thread configuration (not shown).

In some embodiments, the plurality of helical threads may include four helical threads comprising a “quadruple start” or “quadruple lead” thread configuration (not shown).

In some embodiments, the plurality of helical threads may include more than four helical threads (not shown).

In some embodiments, the fastener may include first threading with any of the shapes disclosed herein oriented toward one of the proximal end and the distal end of the fastener, with the first threading located proximate the distal end of the fastener, as well as second threading with any of the shapes disclosed herein oriented toward the other one of the proximal end and the distal end of the fastener, with the second threading located proximate the head of the fastener (not shown).

In some embodiments, the fastener may include multiple threading (e.g., dual helical threading, etc.) with any of the shapes disclosed herein located proximate one of the proximal end and the distal end of the fastener, as well as single threading with any of the shapes disclosed herein with the second threading located proximate the other of the proximal end and the distal end of the fastener.

In some embodiments, the first helical thread 110 may include a plurality of first concave undercut surfaces 131 and a plurality of first convex undercut surfaces 141.

In some embodiments, the second helical thread 120 may include a plurality of second concave undercut surfaces 132 and a plurality of second convex undercut surfaces 142.

In some embodiments, when the fastener is viewed in section along a plane that intersects the longitudinal axis 103 of the shaft 105 (e.g., see FIG. 1D), the plurality of first concave undercut surfaces 131 and the plurality of second convex undercut surfaces 142 may be oriented toward (i.e., point toward) the proximal end 101 of the shaft 105.

In some embodiments, the plurality of first convex undercut surfaces 141 and the plurality of second concave undercut surfaces 132 may be oriented toward (i.e., point toward) the distal end 102 of the shaft 105.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one substantially flat surface.

In some embodiments, when the fastener is viewed in section along a plane intersecting the longitudinal axis 103 of the shaft 105, the first helical thread 110 may comprise a plurality of first bent shapes (comprising at least one surface that is angled relative to the longitudinal axis 103 of the shaft 105 and/or at least one undercut surface) with a plurality of first intermediate portions 151 that are oriented toward (i.e., point toward) the distal end 102 of the shaft 105. This may be referred to as “standard” threading, having a “standard” orientation.

In some embodiments, when the fastener is viewed in section along a plane intersecting the longitudinal axis 103 of the shaft 105, the second helical thread 120 may comprise a plurality of second bent shapes (comprising at least one surface that is angled relative to the longitudinal axis 103 of the shaft 105 and/or at least one undercut surface) with a plurality of second intermediate portions 152 that are oriented toward (i.e., point toward) the proximal end 101 of the shaft 105. This may be referred to as “inverted” threading, having an “inverted” orientation.

In some embodiments, one or more helical threads may morph/transition between a standard orientation and an inverted orientation along a shaft of a fastener.

In some embodiments, at least one of the plurality of first concave undercut surfaces 131, the plurality of first convex undercut surfaces 141, the plurality of second concave undercut surfaces 132, and the plurality of second convex undercut surfaces 142 may comprise at least one curved surface.

As shown in FIG. 1D, the proximally-oriented and distally-oriented surfaces of the first helical thread 110 (i.e., the first concave undercut surfaces 131 and the first convex undercut surfaces 141 in the fastener of FIG. 1D) may not have mirror symmetry relative to each other about any plane perpendicular to the longitudinal axis 103 of the fastener. Rather, the first concave undercut surfaces 131 and the first convex undercut surfaces 141 may be generally parallel to each other. The same may be true for the second helical thread 120, in which the second concave undercut surfaces 132 and the second convex undercut surfaces 142 may not have mirror symmetry relative to each other, but may be generally parallel to each other.

Conversely, as also shown in FIG. 1D, the proximally-oriented surfaces of the first helical thread 110 may have mirror symmetry relative to the distally-oriented surfaces of the second helical thread 120. Specifically, the first concave undercut surfaces 131 may have mirror symmetry relative to the second convex undercut surfaces 142 about a plane 170 that bisects the space between them, and lies perpendicular to the longitudinal axis 103.

Similarly, the distally-oriented surfaces of the first helical thread 110 may have mirror symmetry relative to the proximally-oriented surfaces of the second helical thread 120. Specifically, the second concave undercut surfaces 132 may have mirror symmetry relative to the first convex undercut surfaces 141 about a plane 172 that bisects the space between them, and lies perpendicular to the longitudinal axis 103.

This mirror symmetry may be present along most of the length of the first helical thread 110 and the second helical thread 120, with symmetry across different planes arranged between adjacent turns of the first helical thread 110 and the second helical thread 120 along the length of the longitudinal axis 103. Such mirror symmetry may help more effectively capture bone between the first helical thread 110 and the second helical thread 120 and may also facilitate manufacture of the fastener.

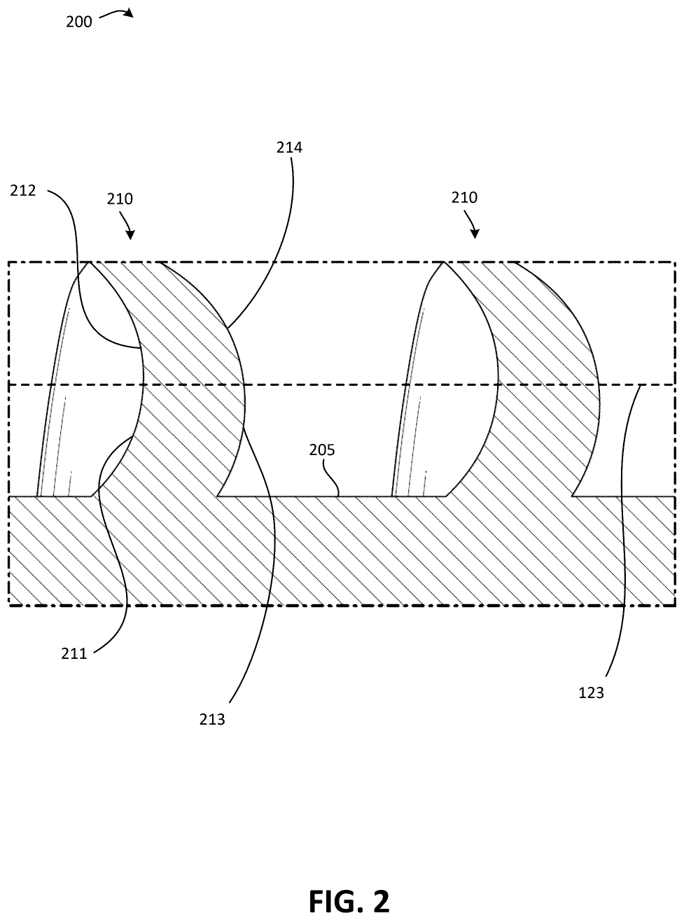

In some embodiments, when the fastener is viewed in section along a plane intersecting the longitudinal axis 103 of the shaft 105, the first helical thread 110 may include at least one partial crescent shape that is oriented toward (i.e., points toward) the distal end 102 of the shaft 105 and/or the proximal end 101 of the shaft 105. FIG. 2 illustrates a partial cross-sectional view of a fastener 200 comprising crescent shapes, as one non-limiting example of such an embodiment.

In some embodiments (not shown), when the fastener is viewed in section along a plane intersecting the longitudinal axis of the shaft, a helical thread 210 may include at least one partial crescent shape that is oriented toward (i.e., points toward) the distal end of the shaft 205, and a second helical thread may include at least one partial crescent shape that is oriented toward (i.e., points toward) the proximal end of the shaft 205.

In some embodiments (not shown), the helical thread may include a first plurality of partial crescent shapes that are oriented toward (i.e., point toward) the distal end of the shaft, and the second helical thread may include a second plurality of partial crescent shapes that are oriented toward (i.e., point toward) the proximal end of the shaft.

In some embodiments (not shown), the first plurality of partial crescent shapes and the second plurality of partial crescent shapes may be arranged in alternating succession along the shaft of the fastener.

In some embodiments, the helical thread 210 may be bisected by the line 123 shown in FIG. 2 with each crescent shape including a plurality of first crescent undercut surfaces 211, a plurality of second crescent undercut surfaces 212, a plurality of third crescent undercut surfaces 213, and a plurality of fourth crescent open surfaces 214 similar to the helical threading shown in FIG. 1D, except with curved surfaces in place of flat surfaces.

In some embodiments, the plurality of first crescent undercut surfaces 211 and the plurality of second crescent undercut surfaces 212 may comprise concave curved surfaces. However, it will be understood that portions of the plurality of first crescent undercut surfaces 211 and/or portions of the plurality of second crescent undercut surfaces 212 may also comprise convex curved surfaces and/or flat surfaces (not shown in FIG. 2).

In some embodiments, the plurality of third crescent undercut surfaces 213 and the plurality of fourth crescent open surfaces 214 may comprise convex curved surfaces. However, it will be understood that portions of the plurality of third crescent undercut surfaces 213 and the plurality of fourth crescent open surfaces 214 may also comprise concave curved surfaces and/or flat surfaces (not shown in FIG. 2).

In some embodiments, the plurality of third crescent undercut surfaces 213 and the plurality of fourth crescent open surfaces 214 may be replaced by a ramped surface (such as that utilized in a standard buttress thread design) without any undercuts (not shown in FIG. 2). Likewise, any of the other thread designs disclosed herein may utilize a ramped or buttress thread design on at least one side of the helical thread.

In some embodiments, a fastener may have only standard threads or only inverted threads. The type of threads that are desired may depend on the type and/or magnitude of loads to be applied to the fastener. For example, a fastener loaded axially away from the bone in which it is implanted may advantageously have a standard thread, while a fastener loaded axially toward the bone in which it is implanted may advantageously have an inverted thread. A fastener that may experience multi-axial loading and/or off-loading conditions may advantageously include at least one standard thread and at least one inverted thread in order to increase bone fixation and load sharing between a bone/fastener interface during multi-axial and off-loading conditions to reduce high bone strain and distribute multi-axial forces applied to the bone in a load-sharing, rather than load-bearing, configuration. Shear loads and/or bending moments may also be optimally resisted with any chosen combination of threading, threading morphology, and/or threading variations contemplated herein to optimally resist shear loads, bending moments, multi-axial loading, off-loading conditions, etc.

In some embodiments, fasteners with standard threads may be used in conjunction with fasteners with inverted threads in order to accommodate different loading patterns.

In some embodiments, a single fastener may have both standard and inverted threads, like the fastener shown in FIGS. 1A-1D. Such a combination of threads may help the fastener remain in place with unknown and/or varying loading patterns.

In some embodiments, the geometry of the threading of a fastener (with standard and/or inverted threads) may be varied to suit the fastener for a particular loading scheme. For example, the number of threads, the number of thread starts, the pitch of the threading, the lead(s) of the threading, the shape(s) of the threading, any dimension(s) associated with the threading (e.g., any length(s)/width(s)/height(s)/inflection point(s), etc., associated with the threading), the major diameter(s), the minor diameter(s), any angulation/angles associated with any surfaces of the threading, the “handedness” of the threading (e.g., right-handed vs. left-handed), etc., may be varied accordingly to suit any specific medium of installation, loading pattern, desired radial loading force, pull-out strength, application, procedure, etc., that may be involved.

In some embodiments, the material(s) of any portion of a fastener, bone implant, joint replacement implant, articular member, interference bone screw, etc., described or contemplated herein may include, but are not limited to: metals (e.g., titanium, cobalt, stainless steel, etc.), metal alloys, plastics, polymers, ceramics, PEEK, UHMWPE, composites, additive particles, textured surfaces, biologics, biomaterials, bio-resorbable materials, bone, etc. Likewise, any fastener, bone implant, joint replacement implant, articular member, interference bone screw, etc., described or contemplated herein may be formed by any manufacturing method including, but not limited to: CNC manufacturing, injection molding, 3D printing, etc., and may include a solid or porous surface to encourage tissue/bone in-growth and/or may include any coating or surface treatment to stimulate tissue/bone growth, exhibit anti-microbial properties, etc.

In some embodiments, any of the fasteners, bone implants, joint replacement implants, articular members, interference bone screws, etc., described herein may include additional features such as: self-tapping features, locking features (e.g., locking threading formed on a portion of the fastener, such as threading located on or near a head of the fastener), opening(s), longitudinal passageways (e.g., to receive a guide wire therethrough), cannulation(s), fenestration(s), any style of fastener head (or no fastener head at all), any style of torque connection interface (or no torque connection interface at all), etc.

In some embodiments, any of the fasteners, bone implants, joint replacement implants, articular members, interference bone screws, etc., described herein may also include opening(s), cannulation(s), fenestration(s), etc., that may be configured to receive any suitable bone cement or bone augment material therein to facilitate bone in-growth, bone fusion, etc.

In some embodiments, a tap tool (not shown) may be utilized to pre-form threading in a bone or bone augment material (or in any other substrate) according to any threading shape that is disclosed or contemplated herein. In this manner, tap tools with any suitable shape may be utilized in conjunction with any fastener described or contemplated herein to match or substantially match the threading geometry of a given fastener, bone implant, interference bone screw, etc.

In some embodiments, a minor diameter of the fastener may be selected to match, or substantially match, a diameter of a pilot hole that is formed in a bone to avoid bone blowout when the fastener is inserted into the pilot hole.

Additionally, or alternatively thereto, the type of threads and/or thread geometry may be varied based on the type of bone in which the fastener is to be anchored. For example, fasteners anchored in osteoporotic bone may fare better with standard or inverted threads, or when the pitch, major diameter, and/or minor diameter are increased or decreased, or when the angulation of thread surfaces is adjusted, etc.

In some embodiments, a surgical kit may include one or more fasteners, bone implants, joint replacement implants, articular members, interference bone screws, etc., that utilize any of the threadforms/features described or contemplated herein, as well as one or more other devices/instruments (e.g., blades, plates, rods, pins, nails, wires, bone screws, etc.). The surgeon may select the appropriate components from the kit based on the particular loads to be applied and/or the quality of bone in which the implant(s) are to be anchored.

Continuing with FIG. 1D, in some embodiments the first helical thread 110 may include a plurality of first undercut surfaces 111, a plurality of second undercut surfaces 112, a plurality of third undercut surfaces 113, a plurality of fourth open surfaces 114,, a plurality of fifth open surfaces 115, a plurality of sixth outer surfaces 116, a first inflection point 117, and a second inflection point 118.

In some embodiments, the second helical thread 120 may include a plurality of fifth undercut surfaces 125, a plurality of sixth undercut surfaces 126, a plurality of seventh undercut surfaces 127, a plurality of eighth open surfaces 128, a plurality of second open surfaces 122, and a plurality of fourth outer surfaces 124.

In some embodiments, one or more of the plurality of first undercut surfaces 111, the plurality of second undercut surfaces 112, the plurality of third undercut surfaces 113, the plurality of fourth open surfaces 114, the plurality of fifth undercut surfaces 125, the plurality of sixth undercut surfaces 126, the plurality of seventh undercut surfaces 127, and the plurality of eighth open surfaces 128 may comprise at least one flat or substantially flat surface.

In some embodiments, the plurality of first undercut surfaces 111, the plurality of third undercut surfaces 113, the plurality of sixth undercut surfaces 126, and the plurality of eighth open surfaces 128 may be angled towards the distal end 102 of the shaft 105.

In some embodiments, the plurality of second undercut surfaces 112, the plurality of fourth open surfaces 114, the plurality of fifth undercut surfaces 125, and the plurality of seventh undercut surfaces 127 may be angled towards the proximal end 101 of the shaft 105.

In some embodiments, when the fastener is viewed in section along a plane that intersects the longitudinal axis 103 of the shaft 105 (as shown in FIG. 1D), the first helical thread 110 may include at least one chevron shape that is oriented toward (i.e., points toward) the distal end 102 of the shaft 105. Likewise, the second helical thread 120 may also include at least one chevron shape that is oriented toward (i.e., points toward) the proximal end 101 of the shaft 105.

In some embodiments, when the fastener is viewed in section along a plane that intersects the longitudinal axis 103 of the shaft 105 (as shown in FIG. 1D), the first helical thread 110 may include a first plurality of chevron shapes that are oriented toward (i.e., point toward) the distal end 102 of the shaft 105. Likewise, the second helical thread 120 may include a second plurality of chevron shapes that are oriented toward (i.e., point toward) the proximal end 101 of the shaft 105.

In some embodiments, the first plurality of chevron shapes and the second plurality of chevron shapes may be arranged in alternating succession along the shaft 105 of the fastener (e.g., see FIG. 1D).

In some embodiments, a plurality of first interlocking spaces 161 and a plurality of second interlocking spaces 162 may be formed between the first helical thread 110 and the second helical thread 120 along the shaft 105 of the fastener.

In some embodiments, the plurality of first interlocking spaces 161 may be formed intermediate the first concave undercut surfaces 131 and the second concave undercut surfaces 132.

In some embodiments, the plurality of second interlocking spaces 162 may be formed intermediate the first convex undercut surfaces 141 and the second convex undercut surfaces 142.

In some embodiments, the plurality of first interlocking spaces 161 may be larger in size than the plurality of second interlocking spaces 162.

In some embodiments, the plurality of first interlocking spaces 161 and the plurality of second interlocking spaces 162 may be shaped and/or configured to interlock with bone/other tissues received therein to increase fixation of the fastener within the bone/other tissues and provide additional resistance against multi-axial forces that may be applied to the fastener and/or the bone/other tissues.

In some embodiments, the plurality of second undercut surfaces 112 and the plurality of sixth undercut surfaces 126 may be angled toward each other to trap bone, soft tissues, other tissues, bone augment material(s), etc., within the plurality of first interlocking spaces 161 in order to increase fixation and resistance against multi-axial forces.

In some embodiments, the plurality of third undercut surfaces 113 and the plurality of seventh undercut surfaces 127 may be angled toward each other to trap bone, soft tissues, other tissues, bone augment material(s), etc., within the plurality of second interlocking spaces 162 in order to increase fixation and resistance against multi-axial forces.

In some embodiments, the plurality of first undercut surfaces 111 and the plurality of fifth undercut surfaces 125 may each form an angle α with respect to the longitudinal axis 103 of the shaft 105, as shown in FIG. 1D.

In some embodiments, the angle a may be greater than 90 degrees.

In some embodiments, the plurality of second undercut surfaces 112 and the plurality of sixth undercut surfaces 126 may each form an angle β with respect to the longitudinal axis 103 of the shaft 105.

In some embodiments, the angle β may be less than 90 degrees.

In some embodiments, the plurality of third undercut surfaces 113 and the plurality of seventh undercut surfaces 127 may each form an angle θ with respect to the longitudinal axis 103 of the shaft 105.

In some embodiments, the angle θ may be approximately 90 degrees.

In some embodiments, the angle θ may be greater than 90 degrees.

It will be understood that any fastener, bone implant, joint replacement implant, articular member, interference bone screw, etc., described or contemplated herein may include any thread configuration, feature, or morphology that is described or contemplated herein with respect to any other fastener, bone implant, joint replacement implant, articular member, interference bone screw, etc., that is described or contemplated herein to achieve optimal fixation within a given bone/tissue for a given soft tissue reconstruction scenario. Moreover, it will also be understood that any fastener, bone implant, joint replacement implant, articular member, interference bone screw, etc., described or contemplated herein may be utilized in conjunction with (or within) any system, method, or instrumentation that is described or contemplated herein.

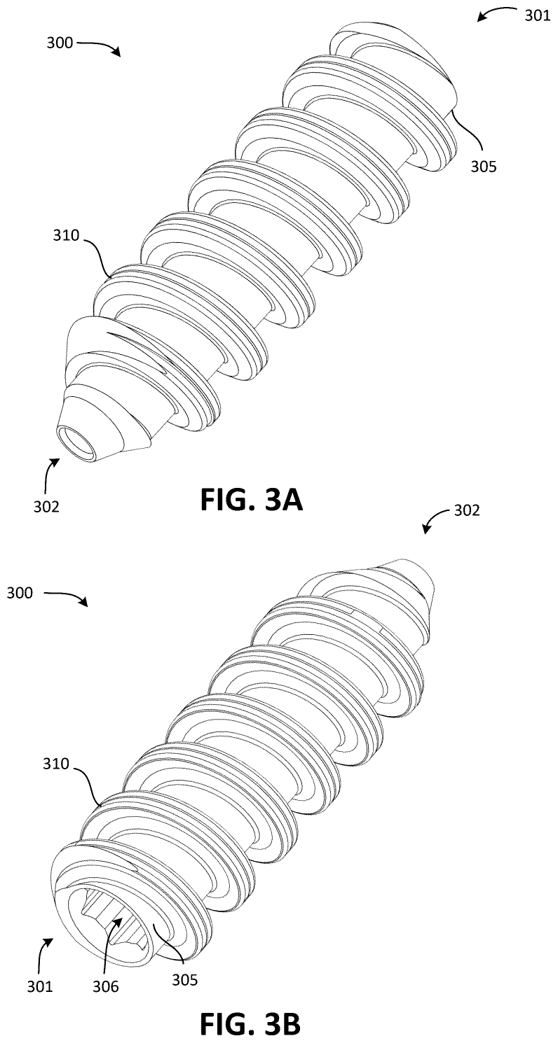

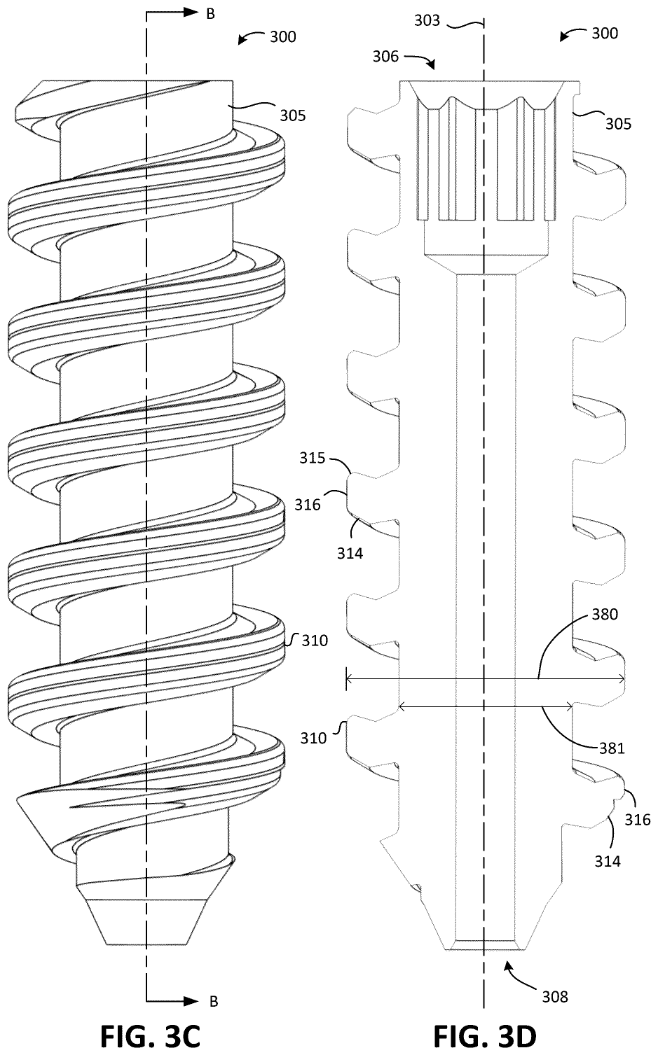

FIGS. 3A-D illustrate various views of a fastener, bone implant, headless screw, or interference bone screw 300, according to another embodiment of the present disclosure. Specifically, FIG. 3A is a perspective distal end view of the interference bone screw 300, FIG. 3B is a perspective proximal end view of the interference bone screw 300, FIG. 3C is a side view of the interference bone screw 300, and FIG. 3D is a cross-sectional side view of the interference bone screw 300 taken along the line B-B in FIG. 3C.

In some embodiments, the interference bone screw 300 may be sized, shaped, and configured for use in soft tissue reconstruction procedures. For example, the interference bone screw 300 may be utilized to directly couple a tendon, ligament, or other soft tissue to a bone, or indirectly couple a tendon, ligament, or other soft tissue to a bone (e.g., via a bone-tendon-bone implant system discussed below). In a particular example, the interference bone screw 300 may be utilized in an ACL reconstruction procedure to either directly, or indirectly, couple a ligament graft to a femoral bone and/or a tibial bone. However, it will also be understood that the interference bone screw 300 may be sized, shaped, and configured for use in any soft tissue graft procedure (e.g., an elbow soft tissue reconstruction procedure, etc.), or for any other procedure or application.

The interference bone screw 300 may generally include a shaft 305 having a proximal end 301, a distal end 302, and a longitudinal axis 303. The interference bone screw 300 may also include a torque connection interface 306 formed in the proximal end 301 of the shaft 305.

In some embodiments, the interference bone screw 300 (and/or any other fastener described or contemplated herein) may include a longitudinal passageway 308 formed through the shaft 305 and configured to receive a guide wire therethrough to help guide placement of the interference bone screw 300 into a bone or a bone tunnel formed in the bone.

In some embodiments, the interference bone screw 300 may also include one or more of the following features: self-tapping features, cutting features, or flutes (of any size, angle, shape, number, or morphology placed along the shaft 305 and/or threading), any style or size of openings, cannulations, fenestrations, or longitudinal/transverse passageways formed in the interference bone screw 300 and configured to receive materials or objects therein (e.g., bone cement/augment materials, guide wires, suture(s), tape(s), etc.), any style of fastener head (or no fastener head at all), any style of torque connection interface (or no torque connection interface at all), etc.

In some embodiments, the distal end 302 of the shaft 305 may comprise a tapered, rounded, or pointed shape to facilitate insertion of the interference bone screw 300 into a bone or a bone tunnel.

In some embodiments, a minor diameter 381 of the interference bone screw 300 may be constant or substantially constant along the entire length of the shaft 305, along a majority of the length of the shaft 305, or along at least a portion of a length of the shaft 305. However, it will also be understood that the minor diameter 381 of the interference bone screw 300 may vary in any fashion (e.g., continuously or discretely increase/decrease, etc.) along the entire length of the shaft 305, along a majority of the length of the shaft 305, or along at least a portion of a length of the shaft 305.

In some embodiments, the minor diameter 381 of the shaft 305 may be configured to decrease in size toward the distal end 302 of the shaft 305 to facilitate insertion of the interference bone screw 300 into a bone tunnel, and/or to provide extra space for bone and/or soft tissues (such as ligaments, tendons, etc.) that may be placed between the distal end 302 of the shaft 305 and the bone tunnel, as will be discussed in more detail below.

In some embodiments, the minor diameter 381 of the shaft 305 may decrease in size toward the distal end 302 of the shaft 305 in a discrete fashion, a continuous fashion, or combinations thereof.

In some embodiments, the interference bone screw 300 may include a single or first helical thread 310 disposed about the shaft 305, as previously discussed herein. In these embodiments, the interference bone screw 300 may comprise a “single start” or “single lead” thread configuration that may include a standard orientation, as shown in FIG. 3D. However, it will also be understood that the interference bone screw 300 may include any thread configuration, feature, or morphology described or contemplated herein to achieve optimal fixation within a given bone or tissue (e.g., an inverted thread configuration, a dual start thread configuration, any thread thickness/height/width/length/pitch/angle/shape, etc.).

In some embodiments, a major diameter 380 of the first helical thread 310 may be constant or substantially constant along the entire length of the first helical thread 310, along a majority of the length of the first helical thread 310, or along at least a portion of a length of the first helical thread 310. However, it will be understood that the major diameter 380 of the first helical thread 310 can vary in any fashion (e.g., continuously or discretely increase/decrease, etc.) along the entire length of the first helical thread 310, along a majority of the length of the first helical thread 310, or along at least a portion of a length of the first helical thread 310.

In some embodiments, the major diameter 380 of the first helical thread 310 may be configured to decrease in size toward the distal end of the first helical thread 310 to facilitate insertion of the interference bone screw 300 into a bone tunnel, and/or to provide extra space for bone/soft tissues that may be placed near the distal end of the first helical thread 310 within the bone tunnel. This configuration may also limit damage to the bone or soft tissues that may be placed near the distal end of the first helical thread 310 (e.g. via cutting as the first helical thread 310 is rotated into place within the bone tunnel, etc.). In this manner, the bone/soft tissues placed near the distal end of the first helical thread 310 may become trapped by, and/or interlock with, the first helical thread 310 to provide increased pull-out resistance for the soft tissue reconstruction implant.

In some embodiments, the major diameter 380 of the first helical thread 310 may decrease in size toward the distal end 302 of the shaft 305 in a discrete fashion, a continuous fashion, or any combination thereof.

In some embodiments, at least one of a fourth open surface 314, a fifth open surface 315, and/or a sixth outer surface 316 of the first helical thread 310 may also be rounded, angled, ground down, or otherwise shaped to further mitigate damage or cutting of the bone/soft tissues by the first helical thread 310 during insertion of the interference bone screw 300 into the bone tunnel (e.g., via rotation, tamping, or combinations thereof).

In some embodiments, a system or kit may include an interference bone screw 300 with standard threading, and an interference bone screw 300 with inverted threading. In some embodiments, a system or kit may include a plurality of interference bone screws 300 of varying sizes with standard threading, and a plurality of interference bone screws 300 of varying sizes with inverted threading. In some embodiments, a surgeon may select an interference bone screw 300 with standard or inverted threading and repair a soft tissue (e.g., a ligament, a tendon, an ACL, etc.) with the selected interference bone screw 300.

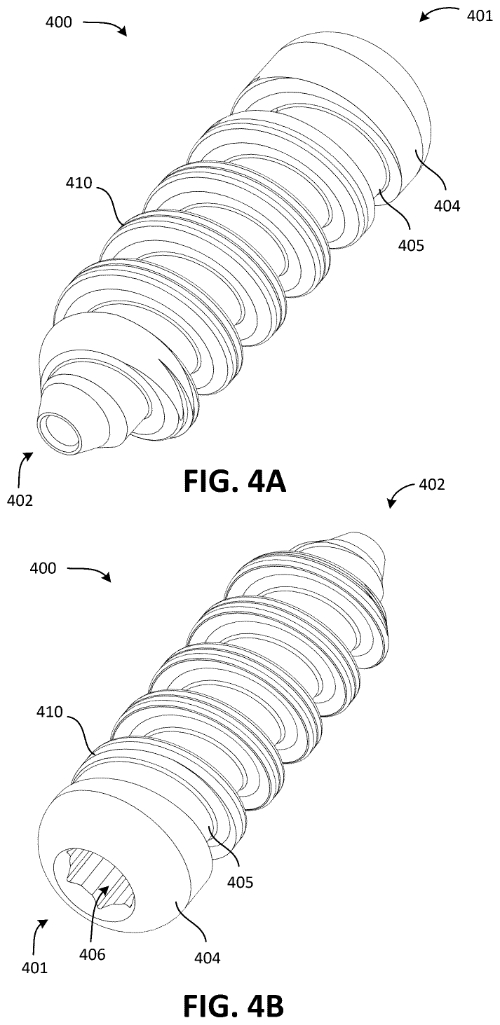

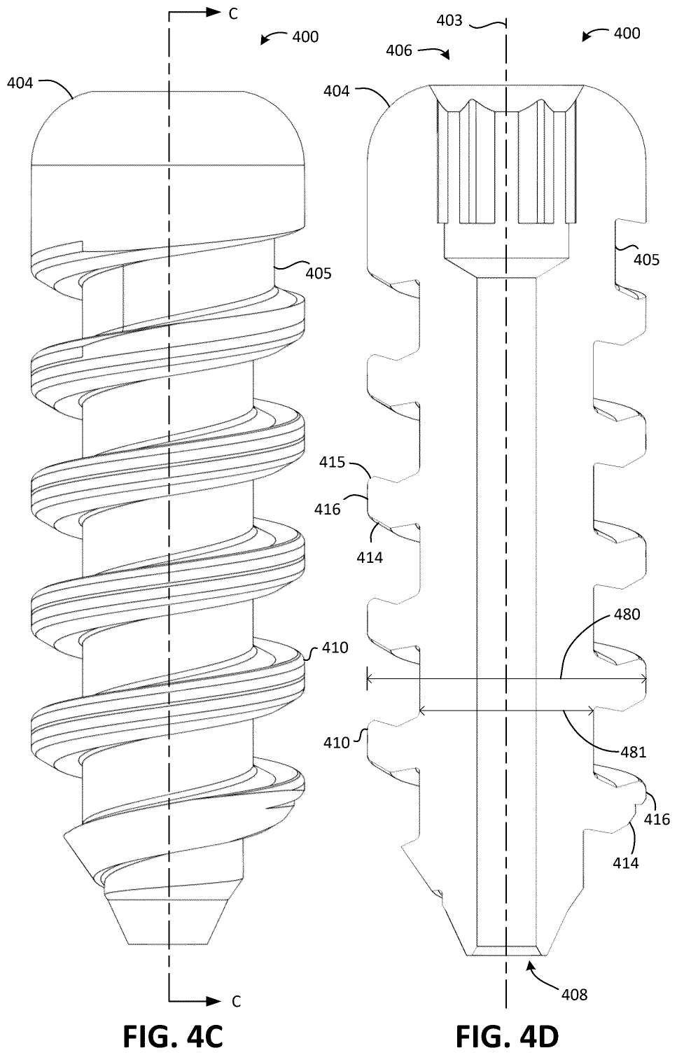

FIGS. 4A-D illustrate various views of a fastener, bone implant, or interference bone screw 400, according to another embodiment of the present disclosure. Specifically, FIG. 4A is a perspective distal end view of the interference bone screw 400, FIG. 4B is a perspective proximal end view of the interference bone screw 400, FIG. 4C is a side view of the interference bone screw 400, and FIG. 4D is a cross-sectional side view of the interference bone screw 400 taken along the line C-C in FIG. 4C.

In some embodiments, the interference bone screw 400 may be sized, shaped, and configured for use in soft tissue reconstruction procedures. For example, the interference bone screw 400 may be utilized to directly couple a tendon, ligament, or other soft tissue to a bone, or indirectly couple a tendon, ligament, or other soft tissue to a bone (e.g., via a bone-tendon-bone implant for an ACL reconstruction procedure, etc.). However, it will also be understood that the interference bone screw 400 may be sized, shaped, and configured for use in any soft tissue graft procedure, or for any other procedure or application.

The interference bone screw 400 may generally include a shaft 405 having a proximal end 401, a distal end 402, and a longitudinal axis 403. The interference bone screw 400 may also include a torque connection interface 406 formed in a head 404 at the proximal end 401 of the shaft 405.

In some embodiments, the interference bone screw 400 (and/or any other fastener described or contemplated herein) may include a longitudinal passageway 408 formed through the shaft 405 and configured to receive a guide wire therethrough to help guide placement of the interference bone screw 400 into a bone or a bone tunnel formed in the bone.

In some embodiments, the interference bone screw 400 may also include one or more of the following features: self-tapping features, cutting features, or flutes (of any size, angle, shape, number, or morphology placed along the shaft 405 and/or threading), any style or size of openings, cannulations, fenestrations, or longitudinal/transverse passageways formed in the interference bone screw 400 and configured to receive materials or objects therein (e.g., bone cement/augment materials, guide wires, suture(s), tape(s), etc.), any style of fastener head (or no fastener head at all), any style of torque connection interface (or no torque connection interface at all), etc.

In some embodiments, the distal end 402 of the shaft 405 may comprise a tapered, rounded, or pointed shape to facilitate insertion of the interference bone screw 400 into a bone or a bone tunnel.

In some embodiments, a minor diameter 481 of the interference bone screw 400 may be constant or substantially constant along the entire length of the shaft 405, along a majority of the length of the shaft 405, or along at least a portion of a length of the shaft 405. However, it will also be understood that the minor diameter 481 of the interference bone screw 400 may vary in any fashion (e.g., continuously or discretely increase/decrease, etc.) along the entire length of the shaft 405, along a majority of the length of the shaft 405, or along at least a portion of a length of the shaft 405.

In some embodiments, the minor diameter 481 of the shaft 405 may be configured to decrease in size toward the distal end 402 of the shaft 405 to facilitate insertion of the interference bone screw 400 into a bone tunnel, and/or to provide extra space for bone and/or soft tissues (such as ligaments, tendons, etc.) that may be placed between the distal end 402 of the shaft 405 and the bone tunnel, as will be discussed in more detail below.

In some embodiments, the minor diameter 481 of the shaft 405 may decrease in size toward the distal end 402 of the shaft 405 in a discrete fashion, a continuous fashion, or combinations thereof.

In some embodiments, the interference bone screw 400 may include a single or first helical thread 410 disposed about the shaft 405, as described herein. In these embodiments, the interference bone screw 400 may comprise a “single start” or “single lead” thread configuration that may include a standard orientation, as shown in FIG. 4D. However, it will also be understood that the interference bone screw 400 may include any thread configuration, feature, or morphology described or contemplated herein to achieve optimal fixation within a given bone or tissue (e.g., an inverted thread configuration, a dual start thread configuration, any thread thickness/height/width/length/pitch/angle/shape, etc.).

In some embodiments, a major diameter 480 of the first helical thread 410 may be constant or substantially constant along the entire length of the first helical thread 410, along a majority of the length of the first helical thread 410, or along at least a portion of a length of the first helical thread 410. However, it will be understood that the major diameter 480 of the first helical thread 410 can vary in any fashion (e.g., continuously or discretely increase/decrease, etc.) along the entire length of the first helical thread 410, along a majority of the length of the first helical thread 410, or along at least a portion of a length of the first helical thread 410.

In some embodiments, the major diameter 480 of the first helical thread 410 may be configured to decrease in size toward the distal end of the first helical thread 410 to facilitate insertion of the interference bone screw 400 into a bone tunnel, and/or to provide extra space for bone/soft tissues that may be placed near the distal end of the first helical thread 410 within the bone tunnel. This configuration may also limit damage to the bone or soft tissues that may be placed near the distal end of the first helical thread 410 (e.g. via cutting as the first helical thread 410 is rotated into place within the bone tunnel, etc.). In this manner, the bone/soft tissues placed near the distal end of the first helical thread 410 may become trapped by, and/or interlock with, the first helical thread 410 to provide increased pull-out resistance for the soft tissue reconstruction implant.

In some embodiments, the major diameter 480 of the first helical thread 410 may decrease in size toward the distal end 402 of the shaft 405 in a discrete fashion, a continuous fashion, or combinations thereof.

In some embodiments, at least one of a fourth open surface 414, a fifth open surface 415, and/or a sixth outer surface 416 of the first helical thread 410 may also be rounded, angled, ground down, or otherwise shaped to further mitigate damage or cutting of the bone/soft tissues by the first helical thread 410 during insertion of the interference bone screw 400 into the bone tunnel (e.g., via rotation, tamping, or combinations thereof).

In some embodiments, a system or kit may include an interference bone screw 400 with standard threading, and an interference bone screw 400 with inverted threading. In some embodiments, a system or kit may include a plurality of interference bone screws 400 of varying sizes with standard threading, and a plurality of interference bone screws 400 of varying sizes with inverted threading. In some embodiments, a surgeon may select an interference bone screw 400 with standard or inverted threading and repair a soft tissue (e.g., a ligament, a tendon, an ACL, etc.) with the selected interference bone screw 400.

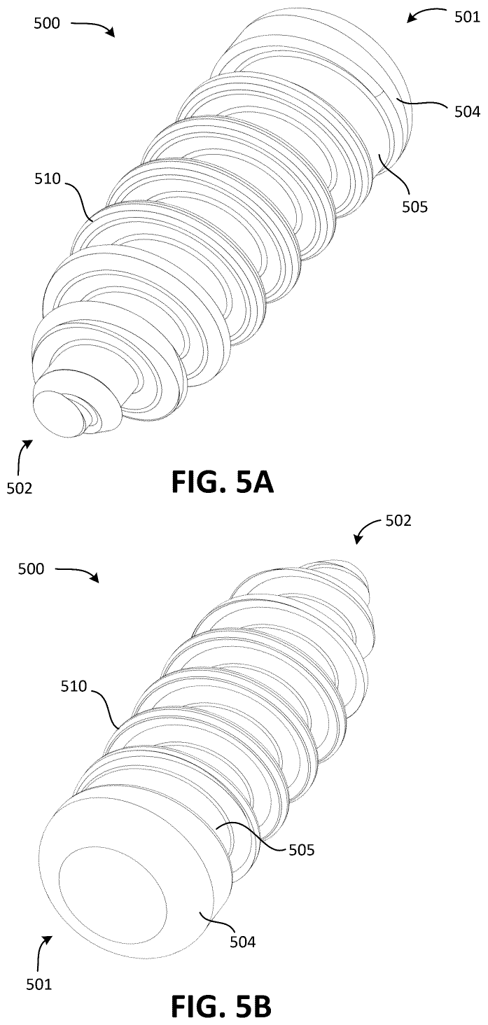

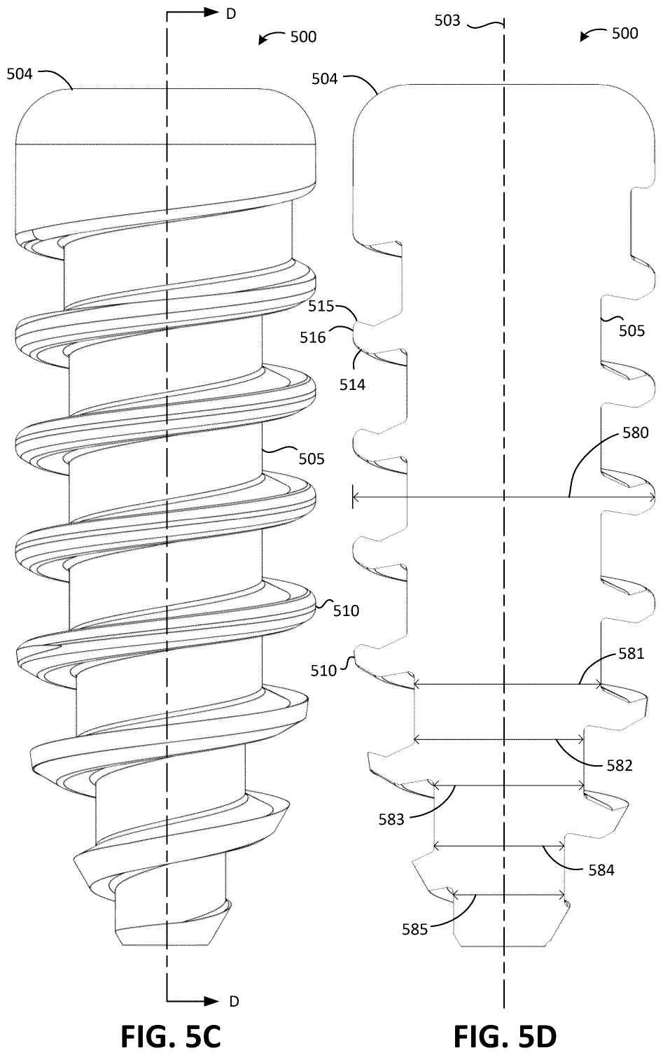

FIGS. 5A-D illustrate various views of a fastener, bone implant, or interference bone screw 500, according to another embodiment of the present disclosure. Specifically, FIG. 5A is a perspective distal end view of the interference bone screw 500, FIG. 5B is a perspective proximal end view of the interference bone screw 500, FIG. 5C is a side view of the interference bone screw 500, and FIG. 5D is a cross-sectional side view of the interference bone screw 500 taken along the line D-D in FIG. 5C.

In some embodiments, the interference bone screw 500 may be sized, shaped, and configured for use in soft tissue reconstruction procedures. For example, the interference bone screw 500 may be utilized to directly couple a tendon, ligament, or other soft tissue to a bone, or indirectly couple a tendon, ligament, or other soft tissue to a bone (e.g., via a bone-tendon-bone implant for an ACL reconstruction procedure, etc.). However, it will also be understood that the interference bone screw 500 may be sized, shaped, and configured for use in any soft tissue graft procedure, or for any other procedure or application.

The interference bone screw 500 may generally include a shaft 505 having a proximal end 501, a distal end 502, and a longitudinal axis 503. The interference bone screw 500 may also include a head 504 at the proximal end 501 of the shaft 505.

In some embodiments, the interference bone screw 500 may also include one or more of the following features: self-tapping features, cutting features, or flutes (of any size, angle, shape, number, or morphology placed along the shaft 505 and/or threading), any style or size of openings, cannulations, fenestrations, or longitudinal/transverse passageways formed in the interference bone screw 500 and configured to receive materials or objects therein (e.g., bone cement/augment materials, guide wires, suture(s), tape(s), etc.), any style of fastener head (or no fastener head at all), any style of torque connection interface (or no torque connection interface at all), etc.

In some embodiments, the distal end 502 of the shaft 505 may comprise a tapered, rounded, or pointed shape to facilitate insertion of the interference bone screw 500 into a bone or a bone tunnel.

In some embodiments, a minor diameter of the interference bone screw 500 may be constant or substantially constant along the entire length of the shaft 505, along a majority of the length of the shaft 505, or along at least a portion of a length of the shaft 505. However, it will also be understood that the minor diameter of the interference bone screw 500 may vary in any fashion (e.g., continuously or discretely increase/decrease, etc.) along the entire length of the shaft 505, along a majority of the length of the shaft 505, or along at least a portion of a length of the shaft 505.

In some embodiments, the minor diameter of the shaft 505 may be configured to decrease in size toward the distal end 502 of the shaft 505 to facilitate insertion of the interference bone screw 500 into a bone tunnel, and/or to provide extra space for bone and/or soft tissues (such as ligaments, tendons, etc.) that may be placed between the distal end 502 of the shaft 505 and the bone tunnel.

In some embodiments, the minor diameter of the shaft 505 may decrease in size toward the distal end 502 of the shaft 505 in a discrete fashion, a continuous fashion, or combinations thereof.

In some embodiments, the interference bone screw 500 may include a plurality of discrete minor diameters. For example, in some embodiments the interference bone screw 500 may include a first minor diameter 581, a second minor diameter 582, a third minor diameter 583, a fourth minor diameter 584, and a fifth minor diameter 585, as shown in FIG. 5D. However, it will be understood that the interference bone screw 500 may include any number of discrete minor diameters.

In some embodiments, the interference bone screw 500 may include a single or first helical thread 510 disposed about the shaft 505, as described herein. In these embodiments, the interference bone screw 500 may comprise a “single start” or “single lead” thread configuration that may include a standard orientation, as shown in FIG. 5D. However, it will also be understood that the interference bone screw 500 may include any thread configuration, feature, or morphology described or contemplated herein to achieve optimal fixation within a given bone or tissue (e.g., an inverted thread configuration, a dual start thread configuration, any thread thickness/height/width/length/pitch/angle/shape, etc.).

In some embodiments, a major diameter 580 of the first helical thread 510 may be constant or substantially constant along the entire length of the first helical thread 510, along a majority of the length of the first helical thread 510, or along at least a portion of a length of the first helical thread 510. However, it will be understood that the major diameter 580 of the first helical thread 510 can vary in any fashion (e.g., continuously or discretely increase/decrease, etc.) along the entire length of the first helical thread 510, along a majority of the length of the first helical thread 510, or along at least a portion of a length of the first helical thread 510.

In some embodiments, the major diameter 580 of the first helical thread 510 may be configured to decrease in size toward the distal end of the first helical thread 510 to facilitate insertion of the interference bone screw 500 into a bone tunnel, and/or to provide extra space for bone or soft tissues that may be placed near the distal end of the first helical thread 510 within the bone tunnel. This configuration may also limit damage to the bone/soft tissues placed near the distal end of the first helical thread 510 (e.g. via cutting as the first helical thread 510 is rotated into place within the bone tunnel, etc.). In this manner, the bone/soft tissues placed near the distal end of the first helical thread 510 may become trapped by, and/or interlock with, the first helical thread 510 to provide increased pull-out resistance for the soft tissue reconstruction implant.

In some embodiments, the major diameter 580 of the first helical thread 510 may decrease in size toward the distal end 502 of the shaft 505 in a discrete fashion, a continuous fashion, or combinations thereof.

In some embodiments, at least one of a fourth open surface 514, a fifth open surface 515, and/or a sixth outer surface 516 of the first helical thread 510 may also be rounded, angled, ground down, or otherwise shaped to further mitigate damage or cutting of the bone/soft tissues by the first helical thread 510 during insertion of the interference bone screw 500 into the bone tunnel (e.g., via rotation, tamping, or combinations thereof).

In some embodiments, a system or kit may include an interference bone screw 500 with standard threading, and an interference bone screw 500 with inverted threading. In some embodiments, a system or kit may include a plurality of interference bone screws 500 of varying sizes with standard threading, and a plurality of interference bone screws 500 of varying sizes with inverted threading. In some embodiments, a surgeon may select an interference bone screw 500 with standard or inverted threading and repair a soft tissue (e.g., a ligament, a tendon, an ACL, etc.) with the selected interference bone screw 500.

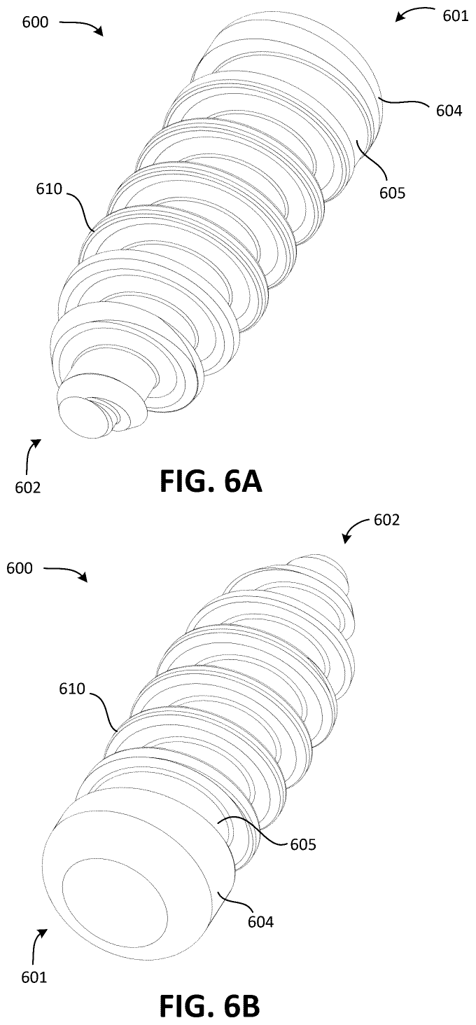

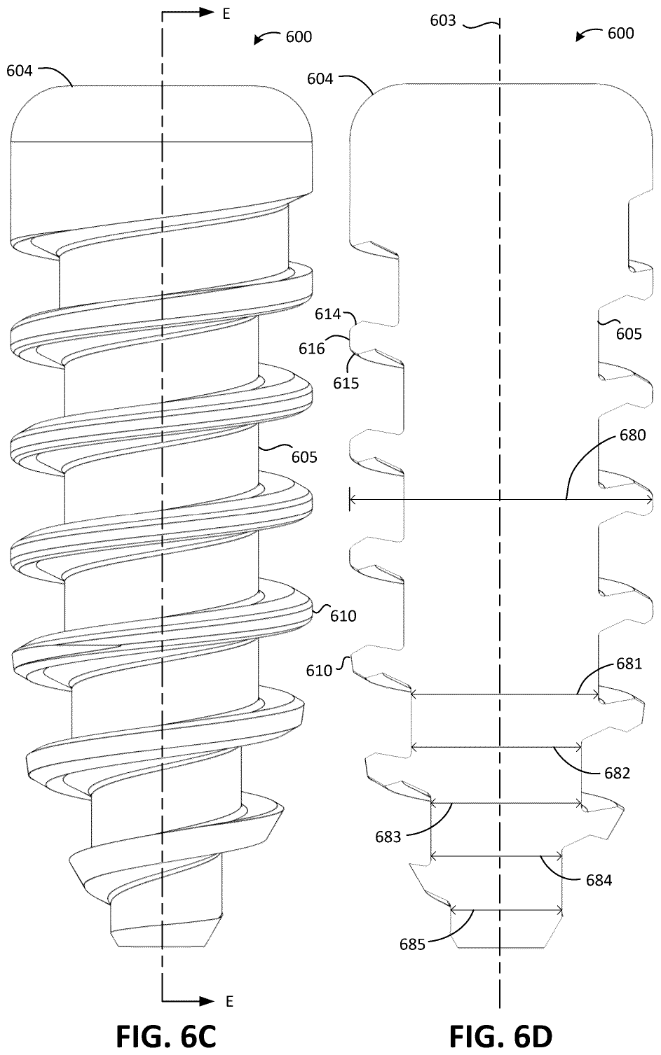

FIGS. 6A-D illustrate various views of a fastener, bone implant, or interference bone screw 600, according to another embodiment of the present disclosure. Specifically, FIG. 6A is a perspective distal end view of the interference bone screw 600, FIG. 6B is a perspective proximal end view of the interference bone screw 600, FIG. 6C is a side view of the interference bone screw 600, and FIG. 6D is a cross-sectional side view of the interference bone screw 600 taken along the line E-E in FIG. 6C.

In some embodiments, the interference bone screw 600 may be sized, shaped, and configured for use in soft tissue reconstruction procedures. For example, the interference bone screw 600 may be utilized to directly couple a tendon, ligament, or other soft tissue to a bone, or indirectly couple a tendon, ligament, or other soft tissue to a bone (e.g., via a bone-tendon-bone implant for an ACL reconstruction procedure, etc.). However, it will also be understood that the interference bone screw 600 may be sized, shaped, and configured for use in any soft tissue graft procedure, or for any other procedure or application.

The interference bone screw 600 may generally include a shaft 605 having a proximal end 601, a distal end 602, and a longitudinal axis 603. The interference bone screw 600 may also include a head 604 at the proximal end 601 of the shaft 605.

In some embodiments, the interference bone screw 600 may also include one or more of the following features: self-tapping features, cutting features, or flutes (of any size, angle, shape, number, or morphology placed along the shaft 605 and/or threading), any style or size of openings, cannulations, fenestrations, or longitudinal/transverse passageways formed in the interference bone screw 600 and configured to receive materials or objects therein (e.g., bone cement/augment materials, guide wires, suture(s), tape(s), etc.), any style of fastener head (or no fastener head at all), any style of torque connection interface (or no torque connection interface at all), etc.

In some embodiments, the distal end 602 of the shaft 605 may comprise a tapered, rounded, or pointed shape to facilitate insertion of the interference bone screw 600 into a bone or a bone tunnel.

In some embodiments, a minor diameter of the interference bone screw 600 may be constant or substantially constant along the entire length of the shaft 605, along a majority of the length of the shaft 605, or along at least a portion of a length of the shaft 605. However, it will also be understood that the minor diameter of the interference bone screw 600 may vary in any fashion (e.g., continuously or discretely increase/decrease, etc.) along the entire length of the shaft 605, along a majority of the length of the shaft 605, or along at least a portion of a length of the shaft 605.

In some embodiments, the minor diameter of the shaft 605 may be configured to decrease in size toward the distal end 602 of the shaft 605 to facilitate insertion of the interference bone screw 600 into a bone tunnel, and/or to provide extra space for bone and/or soft tissues (such as ligaments, tendons, etc.) that may be placed between the distal end 602 of the shaft 605 and the bone tunnel.

In some embodiments, the minor diameter of the shaft 605 may decrease in size toward the distal end 602 of the shaft 605 in a discrete fashion, a continuous fashion, or combinations thereof.

In some embodiments, the interference bone screw 600 may include a plurality of discrete minor diameters. For example, in some embodiments the interference bone screw 600 may include a first minor diameter 681, a second minor diameter 682, a third minor diameter 683, a fourth minor diameter 684, and a fifth minor diameter 685, as shown in FIG. 6D. However, it will be understood that the interference bone screw 600 may include any number of discrete minor diameters.

In some embodiments, the interference bone screw 600 may include a single or first helical thread 610 disposed about the shaft 605, as described herein. In these embodiments, the interference bone screw 600 may comprise a “single start” or “single lead” thread configuration that may include an inverted orientation, as shown in FIG. 6D. However, it will also be understood that the interference bone screw 600 may include any thread configuration, feature, or morphology described or contemplated herein to achieve optimal fixation within a given bone or tissue (e.g., a standard thread configuration, a start dual thread configuration, any thread thickness/height/width/length/pitch/angle/shape, etc.).

In some embodiments, a major diameter 680 of the first helical thread 610 may be constant or substantially constant along the entire length of the first helical thread 610, along a majority of the length of the first helical thread 610, or along at least a portion of a length of the first helical thread 610. However, it will be understood that the major diameter 680 of the first helical thread 610 can vary in any fashion (e.g., continuously or discretely increase/decrease, etc.) along the entire length of the first helical thread 610, along a majority of the length of the first helical thread 610, or along at least a portion of a length of the first helical thread 610.

In some embodiments, the major diameter 680 of the first helical thread 610 may be configured to decrease in size toward the distal end of the first helical thread 610 to facilitate insertion of the interference bone screw 600 into a bone tunnel, and/or to provide extra space for bone or soft tissues that may be placed near the distal end of the first helical thread 610 within the bone tunnel. This configuration may also limit damage to the bone/soft tissues placed near the distal end of the first helical thread 610 (e.g. via cutting as the first helical thread 610 is rotated into place within the bone tunnel, etc.). In this manner, the bone/soft tissues placed near the distal end of the first helical thread 610 may become trapped by, and/or interlock with, the first helical thread 610 to provide increased pull-out resistance for the soft tissue reconstruction implant.

In some embodiments, the major diameter 680 of the first helical thread 610 may decrease in size toward the distal end 602 of the shaft 605 in a discrete fashion, a continuous fashion, or combinations thereof.

In some embodiments, at least one of a fourth open surface 614, a fifth open surface 615, and/or a sixth outer surface 616 of the first helical thread 610 may also be rounded, angled, ground down, or otherwise shaped to further mitigate damage or cutting of the bone/soft tissues by the first helical thread 610 during insertion of the interference bone screw 600 into the bone tunnel (e.g., via rotation, tamping, or combinations thereof).

In some embodiments, a system or kit may include an interference bone screw 600 with standard threading, and an interference bone screw 600 with inverted threading. In some embodiments, a system or kit may include a plurality of interference bone screws 600 of varying sizes with standard threading, and a plurality of interference bone screws 600 of varying sizes with inverted threading. In some embodiments, a surgeon may select an interference bone screw 600 with standard or inverted threading and repair a soft tissue (e.g., a ligament, a tendon, an ACL, etc.) with the selected interference bone screw 600.

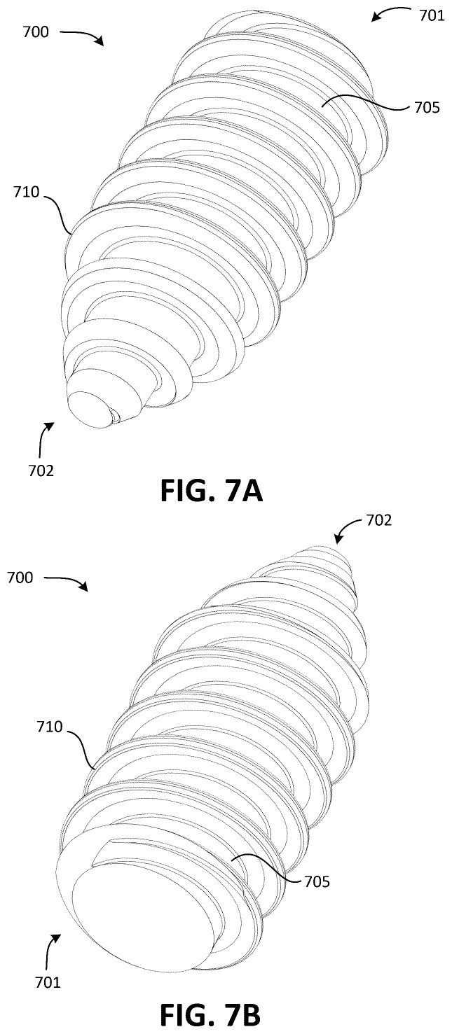

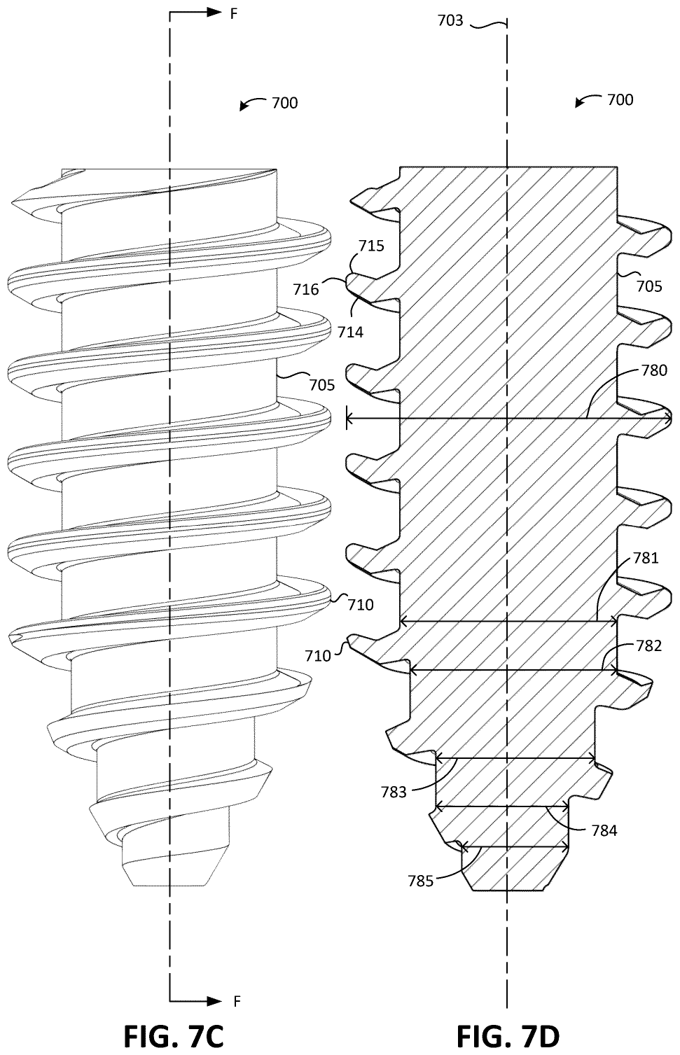

FIGS. 7A-D illustrate various views of a fastener, bone implant, or interference bone screw 700, according to another embodiment of the present disclosure. Specifically, FIG. 7A is a front perspective view of the interference bone screw 700, FIG. 7B is a rear perspective view of the interference bone screw 700, FIG. 7C is a side view of the interference bone screw 700, and FIG. 7D is a cross-sectional side view of the interference bone screw 700 taken along the line F-F in FIG. 7C.

In some embodiments, the interference bone screw 700 may be sized, shaped, and configured for use in soft tissue reconstruction procedures. For example, the interference bone screw 700 may be utilized to directly couple a tendon, ligament, or other soft tissue to a bone, or indirectly couple a tendon, ligament, or other soft tissue to a bone (e.g., via a bone-tendon-bone implant for an ACL reconstruction procedure, etc.). However, it will also be understood that the interference bone screw 700 may be sized, shaped, and configured for use in any soft tissue graft procedure, or for any other procedure or application.

The interference bone screw 700 may generally include a shaft 705 having a proximal end 701, a distal end 702, and a longitudinal axis 703.

In some embodiments, the interference bone screw 700 may also include one or more of the following features: self-tapping features, cutting features, or flutes (of any size, angle, shape, number, or morphology placed along the shaft 705 and/or threading), any style or size of openings, cannulations, fenestrations, or longitudinal/transverse passageways formed in the interference bone screw 700 and configured to receive materials or objects therein (e.g., bone cement/augment materials, guide wires, suture(s), tape(s), etc.), any style of fastener head (or no fastener head at all), any style of torque connection interface (or no torque connection interface at all), etc.

In some embodiments, the distal end 702 of the shaft 705 may comprise a tapered, rounded, or pointed shape to facilitate insertion of the interference bone screw 700 into a bone or a bone tunnel.

In some embodiments, a minor diameter of the interference bone screw 700 may be constant or substantially constant along the entire length of the shaft 705, along a majority of the length of the shaft 705, or along at least a portion of a length of the shaft 705. However, it will also be understood that the minor diameter of the interference bone screw 700 may vary in any fashion (e.g., continuously or discretely increase/decrease, etc.) along the entire length of the shaft 705, along a majority of the length of the shaft 705, or along at least a portion of a length of the shaft 705.

In some embodiments, the minor diameter of the shaft 705 may be configured to decrease in size toward the distal end 702 of the shaft 705 to facilitate insertion of the interference bone screw 700 into a bone tunnel, and/or to provide extra space for bone and/or soft tissues (such as ligaments, tendons, etc.) that may be placed between the distal end 702 of the shaft 705 and the bone tunnel.

In some embodiments, the minor diameter of the shaft 705 may decrease in size toward the distal end 702 of the shaft 705 in a discrete fashion, a continuous fashion, or combinations thereof.

In some embodiments, the interference bone screw 700 may include a plurality of discrete minor diameters. For example, in some embodiments the interference bone screw 700 may include a first minor diameter 781, a second minor diameter 782, a third minor diameter 783, a fourth minor diameter 784, and a fifth minor diameter 785, as shown in FIG. 7D. However, it will be understood that the interference bone screw 700 may include any number of discrete minor diameters.

In some embodiments, the interference bone screw 700 may include a single or first helical thread 710 disposed about the shaft 705, as described herein. In these embodiments, the interference bone screw 700 may comprise a “single start” or “single lead” thread configuration that may include a standard orientation, as shown in FIG. 7D. However, it will also be understood that the interference bone screw 700 may include any thread configuration, feature, or morphology described or contemplated herein to achieve optimal fixation within a given bone or tissue (e.g., an inverted thread configuration, a dual start thread configuration, any thread thickness/height/width/length/pitch/angle/shape, etc.).

In some embodiments, a major diameter 780 of the first helical thread 710 may be constant or substantially constant along the entire length of the first helical thread 710, along a majority of the length of the first helical thread 710, or along at least a portion of a length of the first helical thread 710. However, it will be understood that the major diameter 780 of the first helical thread 710 can vary in any fashion (e.g., continuously or discretely increase/decrease, etc.) along the entire length of the first helical thread 710, along a majority of the length of the first helical thread 710, or along at least a portion of a length of the first helical thread 710.

In some embodiments, the major diameter 780 of the first helical thread 710 may be configured to decrease in size toward the distal end of the first helical thread 710 to facilitate insertion of the interference bone screw 700 into a bone tunnel, and/or to provide extra space for bone or soft tissues that may be placed near the distal end of the first helical thread 710 within the bone tunnel. This configuration may also limit damage to the bone/soft tissues placed near the distal end of the first helical thread 710 (e.g. via cutting as the first helical thread 710 is rotated into place within the bone tunnel, etc.). In this manner, the bone/soft tissues placed near the distal end of the first helical thread 710 may become trapped by, and/or interlock with, the first helical thread 710 to provide increased pull-out resistance for the soft tissue reconstruction implant.

In some embodiments, the major diameter 780 of the first helical thread 710 may decrease in size toward the distal end 702 of the shaft 705 in a discrete fashion, a continuous fashion, or combinations thereof.

In some embodiments, at least one of a fourth open surface 714, a fifth open surface 715, and/or a sixth outer surface 716 of the first helical thread 710 may also be rounded, angled, ground down, or otherwise shaped to further mitigate damage or cutting of the bone/soft tissues by the first helical thread 710 during insertion of the interference bone screw 700 into the bone tunnel (e.g., via rotation, tamping, or combinations thereof).

In some embodiments, a system or kit may include an interference bone screw 700 with standard threading, and an interference bone screw 700 with inverted threading. In some embodiments, a system or kit may include a plurality of interference bone screws 700 of varying sizes with standard threading, and a plurality of interference bone screws 700 of varying sizes with inverted threading. In some embodiments, a surgeon may select an interference bone screw 700 with standard or inverted threading and repair a soft tissue (e.g., a ligament, a tendon, an ACL, etc.) with the selected interference bone screw 700.

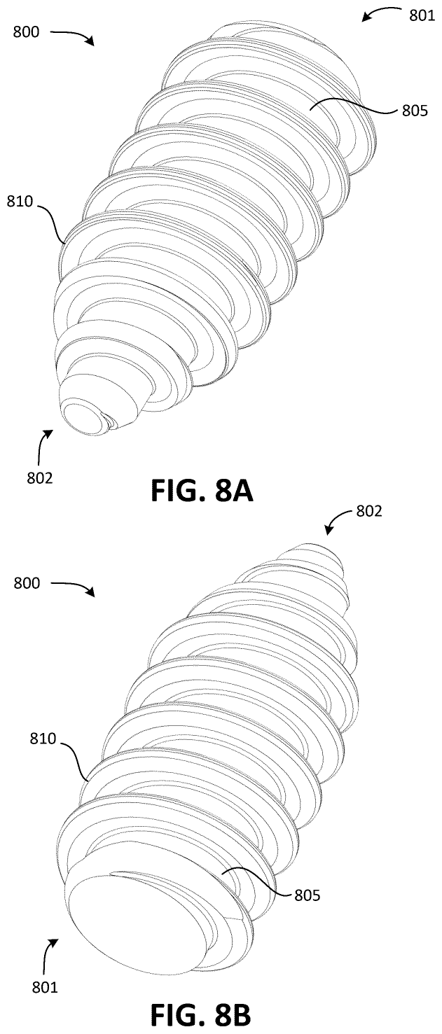

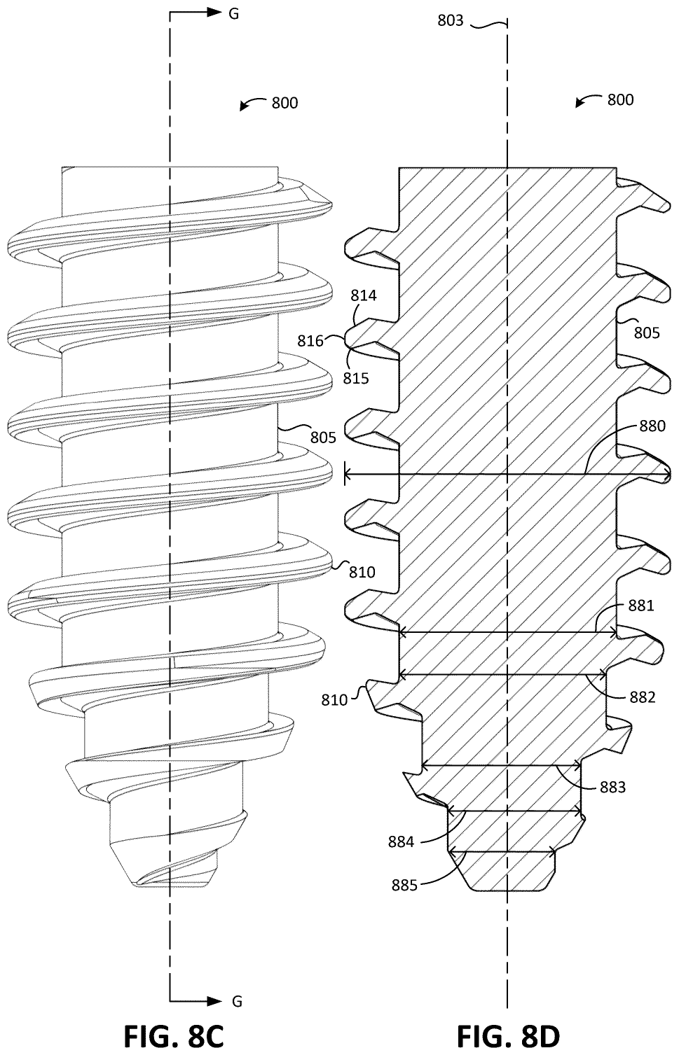

FIGS. 8A-D illustrate various views of a fastener, bone implant, or interference bone screw 800, according to another embodiment of the present disclosure. Specifically, FIG. 8A is a front perspective view of the interference bone screw 800, FIG. 8B is a rear perspective view of the interference bone screw 800, FIG. 8C is a side view of the interference bone screw 800, and FIG. 8D is a cross-sectional side view of the interference bone screw 800 taken along the line G-G in FIG. 8C.

In some embodiments, the interference bone screw 800 may be sized, shaped, and configured for use in soft tissue reconstruction procedures. For example, the interference bone screw 800 may be utilized to directly couple a tendon, ligament, or other soft tissue to a bone, or indirectly couple a tendon, ligament, or other soft tissue to a bone (e.g., via a bone-tendon-bone implant for an ACL reconstruction procedure, etc.). However, it will also be understood that the interference bone screw 800 may be sized, shaped, and configured for use in any soft tissue graft procedure, or for any other procedure or application.

The interference bone screw 800 may generally include a shaft 805 having a proximal end 801, a distal end 802, and a longitudinal axis 803.

In some embodiments, the interference bone screw 800 may also include one or more of the following features: self-tapping features, cutting features, or flutes (of any size, angle, shape, number, or morphology placed along the shaft 805 and/or threading), any style or size of openings, cannulations, fenestrations, or longitudinal/transverse passageways formed in the interference bone screw 800 and configured to receive materials or objects therein (e.g., bone cement/augment materials, guide wires, suture(s), tape(s), etc.), any style of fastener head (or no fastener head at all), any style of torque connection interface (or no torque connection interface at all), etc.

In some embodiments, the distal end 802 of the shaft 805 may comprise a tapered, rounded, or pointed shape to facilitate insertion of the interference bone screw 800 into a bone or a bone tunnel.

In some embodiments, a minor diameter of the interference bone screw 800 may be constant or substantially constant along the entire length of the shaft 805, along a majority of the length of the shaft 805, or along at least a portion of a length of the shaft 805. However, it will also be understood that the minor diameter of the interference bone screw 800 may vary in any fashion (e.g., continuously or discretely increase/decrease, etc.) along the entire length of the shaft 805, along a majority of the length of the shaft 805, or along at least a portion of a length of the shaft 805.

In some embodiments, the minor diameter of the shaft 805 may be configured to decrease in size toward the distal end 802 of the shaft 805 to facilitate insertion of the interference bone screw 800 into a bone tunnel, and/or to provide extra space for bone and/or soft tissues (such as ligaments, tendons, etc.) that may be placed between the distal end 802 of the shaft 805 and the bone tunnel.

In some embodiments, the minor diameter of the shaft 805 may decrease in size toward the distal end 802 of the shaft 805 in a discrete fashion, a continuous fashion, or combinations thereof.

In some embodiments, the interference bone screw 800 may include a plurality of discrete minor diameters. For example, in some embodiments the interference bone screw 800 may include a first minor diameter 881, a second minor diameter 882, a third minor diameter 883, a fourth minor diameter 884, and a fifth minor diameter 885, as shown in FIG. 8D. However, it will be understood that the interference bone screw 800 may include any number of discrete minor diameters.

In some embodiments, the interference bone screw 800 may include a single or first helical thread 810 disposed about the shaft 805, as described herein. In these embodiments, the interference bone screw 800 may comprise a “single start” or “single lead” thread configuration that may include an inverted orientation, as shown in FIG. 8D. However, it will also be understood that the interference bone screw 800 may include any thread configuration, feature, or morphology described or contemplated herein to achieve optimal fixation within a given bone or tissue (e.g., a standard thread configuration, a dual start thread configuration, any thread thickness/height/width/length/pitch/angle/shape, etc.).

In some embodiments, a major diameter 880 of the first helical thread 810 may be constant or substantially constant along the entire length of the first helical thread 810, along a majority of the length of the first helical thread 810, or along at least a portion of a length of the first helical thread 810. However, it will be understood that the major diameter 880 of the first helical thread 810 can vary in any fashion (e.g., continuously or discretely increase/decrease, etc.) along the entire length of the first helical thread 810, along a majority of the length of the first helical thread 810, or along at least a portion of a length of the first helical thread 810.

In some embodiments, the major diameter 880 of the first helical thread 810 may be configured to decrease in size toward the distal end of the first helical thread 810 to facilitate insertion of the interference bone screw 800 into a bone tunnel, and/or to provide extra space for bone or soft tissues that may be placed near the distal end of the first helical thread 810 within the bone tunnel. This configuration may also limit damage to the bone/soft tissues placed near the distal end of the first helical thread 810 (e.g. via cutting as the first helical thread 810 is rotated into place within the bone tunnel, etc.). In this manner, the bone/soft tissues placed near the distal end of the first helical thread 810 may become trapped by, and/or interlock with, the first helical thread 810 to provide increased pull-out resistance for the soft tissue reconstruction implant.

In some embodiments, the major diameter 880 of the first helical thread 810 may decrease in size toward the distal end 802 of the shaft 805 in a discrete fashion, a continuous fashion, or combinations thereof.

In some embodiments, at least one of a fourth open surface 814, a fifth open surface 815, and/or a sixth outer surface 816 of the first helical thread 810 may also be rounded, angled, ground down, or otherwise shaped to further mitigate damage or cutting of the bone/soft tissues by the first helical thread 810 during insertion of the interference bone screw 800 into the bone tunnel (e.g., via rotation, tamping, or combinations thereof).

In some embodiments, a system or kit may include an interference bone screw 800 with standard threading, and an interference bone screw 800 with inverted threading. In some embodiments, a system or kit may include a plurality of interference bone screws 800 of varying sizes with standard threading, and a plurality of interference bone screws 800 of varying sizes with inverted threading. In some embodiments, a surgeon may select an interference bone screw 800 with standard or inverted threading and repair a soft tissue (e.g., a ligament, a tendon, an ACL, etc.) with the selected interference bone screw 800.

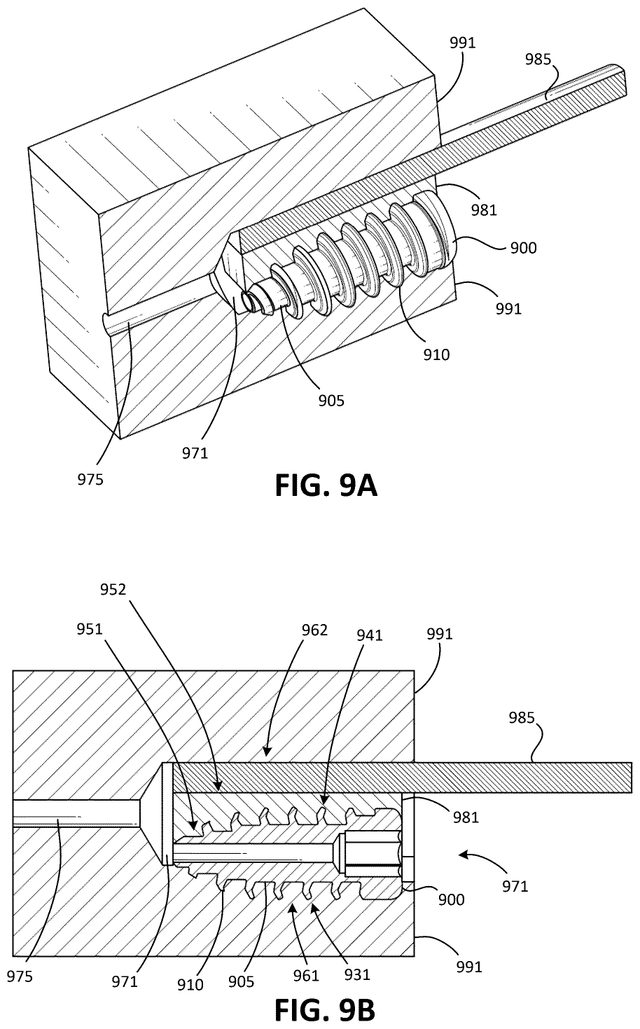

FIGS. 9A and 9B illustrate a system or procedure for reconstructing a soft tissue, according to an embodiment of the present disclosure. FIG. 9A shows a perspective view of the system and FIG. 9B shows a cross-sectional side view of the system.

The system may generally include a first interference bone screw 900 and a soft tissue implant 985 (e.g., a soft tissue, a tendon, a ligament, a synthetic/artificial ligament, etc.) that is couplable within a first bone tunnel 971 formed in a first bone 991 and held in place with the first interference bone screw 900 within the first bone tunnel 971. The first interference bone screw 900 may comprise any interference bone screw embodiment that is described or contemplated herein.

In some embodiments, a pilot hole or guide wire tunnel 975 may be formed in the first bone 991 via a drill tool (not shown) or via a guide wire (not shown) that may be placed within the first bone 991.

In some embodiments, the first bone tunnel 971 may be formed in the first bone 991 via a drill tool (not shown) and sized/shaped to receive at least the soft tissue implant 985 and the first interference bone screw 900 therein.

In some embodiments, a tap tool (not shown) may also be utilized to tap at least one side or a first side 961 of the first bone tunnel 971 to form a first bone tunnel pre-formed thread 931 in the first bone tunnel 971 that may be configured to receive at least a portion of the first helical thread 910 therein. However, it will also be understood that, in a least some embodiments, the first interference bone screw 900 may comprise self-tapping features, cutting features, and/or flutes that may obviate the need for a tap tool.

In some embodiments, the soft tissue implant 985 may be placed within the first bone tunnel 971, and the first interference bone screw 900 may be inserted into the first bone tunnel 971 to interlock the first helical thread 910 with the first bone 991 and/or with the soft tissue implant 985 to couple the first interference bone screw 900 with the first bone 991, as well as press/trap/interlock the soft tissue implant 985 within first bone tunnel 971 intermediate the threads of the first interference bone screw 900. In this manner, the first helical thread 910 may interlock with the first side 961 of the first bone tunnel 971 to couple the first interference bone screw 900 to the first bone 991, as well as directly engage the soft tissue implant 985 to press/trap/interlock the soft tissue implant 985 within the first helical thread 910 and/or against a second side 962 of the first bone tunnel 971 to couple the soft tissue implant 985 within the first bone tunnel 971.

In some embodiments, the system may also include a bone block or first bone block 981 that may be coupled to the soft tissue implant 985 and placed intermediate the first interference bone screw 900 and the soft tissue implant 985 within the first bone tunnel 971, as shown in FIGS. 9A and 9B.

In these embodiments, a tap tool (not shown) may be utilized to tap a first side 951 of the first bone block 981 to form a first pre-formed thread 941 in the first bone block 981 that may be configured to receive at least a portion of the first helical thread 910 therein. However, it will also be understood that, in a least some embodiments, the first interference bone screw 900 may comprise self-tapping features, cutting features, and/or flutes that may obviate the need for a tap tool.

In this manner, the first helical thread 910 may interlock with the first side 961 of the first bone tunnel 971 and the first side 951 of the first bone block 981 to couple the first interference bone screw 900 to the first bone 991, while pressing the soft tissue implant 985 against the second side 962 of the first bone tunnel 971 with the second side 952 of the first bone block 981 to couple the soft tissue implant 985 within the first bone tunnel 971.

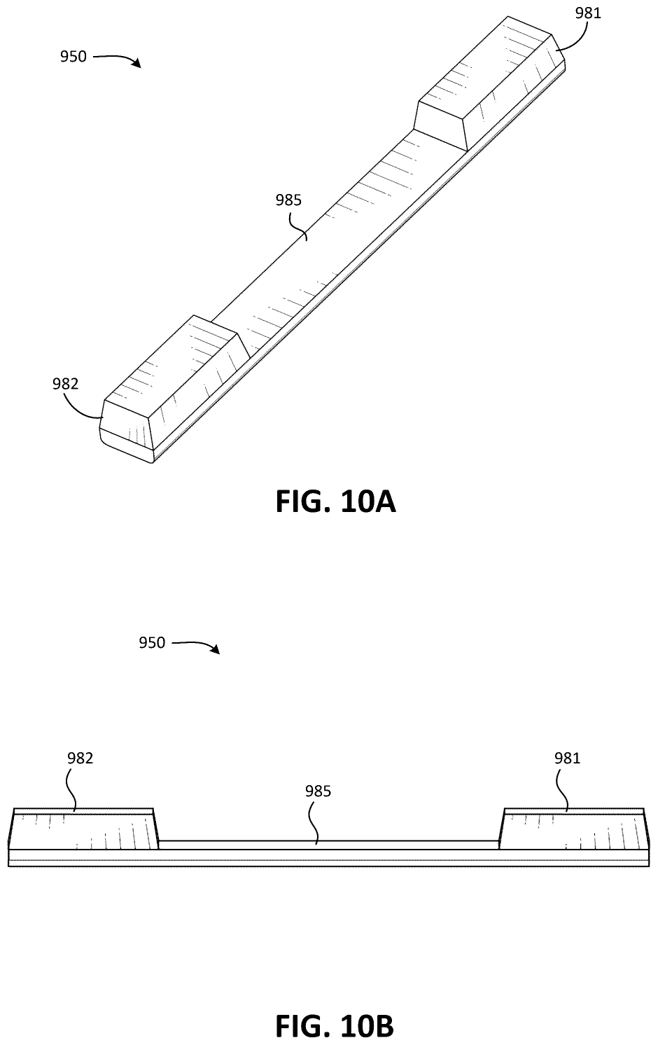

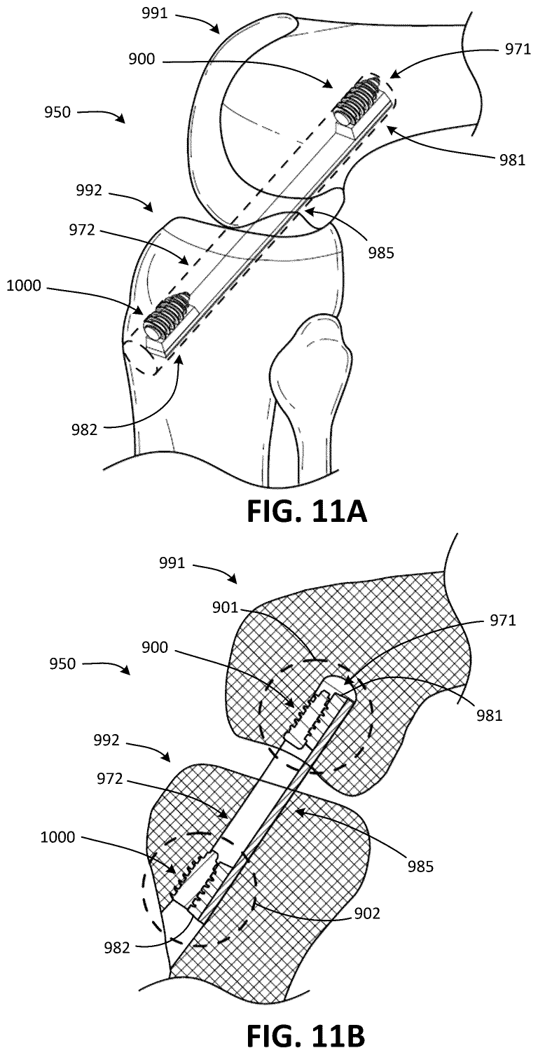

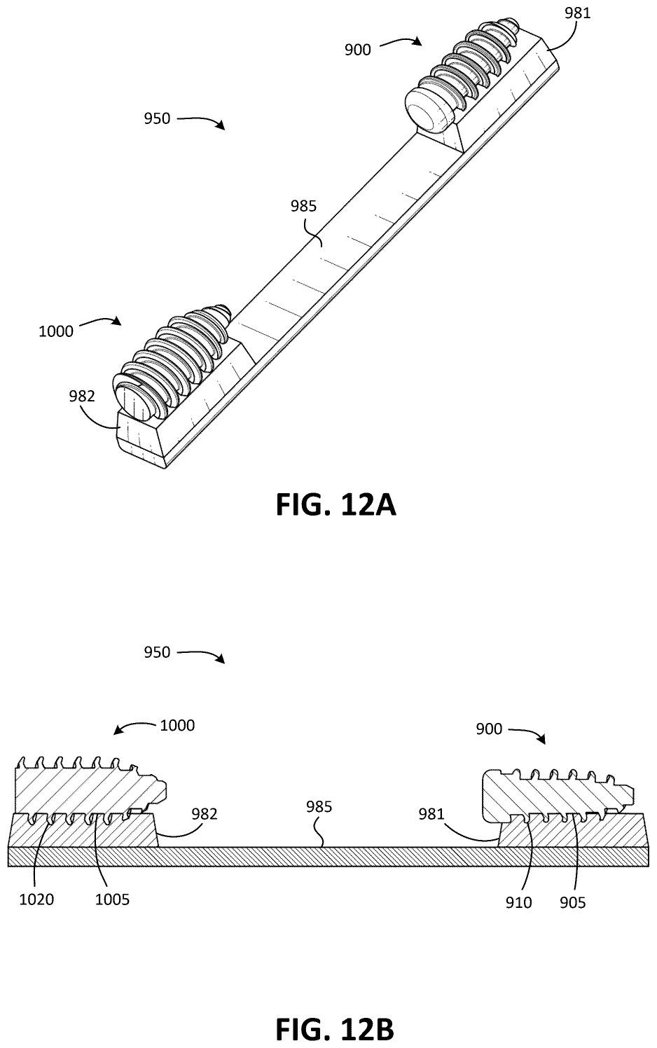

FIGS. 10A-15B illustrate various systems and procedures for reconstructing a soft tissue, according to some embodiments of the present disclosure. Specifically, FIGS. 10A and 10B show a soft tissue implant or bone-tendon-bone implant 950 that may be utilized with the various systems described herein; FIGS. 11A and 11B show an example system installed in a knee joint to reconstruct an ACL; and FIGS. 12A-15B show various systems utilizing different interference bone screw embodiments to reconstruct a soft tissue, according to some embodiments of the present disclosure.

With reference to FIGS. 10A and 10B, in some embodiments the bone-tendon-bone implant 950 may include a first bone block 981, a second bone block 982, and a soft tissue implant 985 coupled intermediate the first bone block 981 and the second bone block 982.

In some embodiments, the soft tissue implant 985 may comprise any soft tissue (e.g., such as a tendon, a ligament, etc.), or any synthetic/artificial ligament material(s), or any combinations thereof.

In some embodiments, the first bone block 981 and/or the second bone block 982 may comprise any bone material(s), or any synthetic/artificial bone material(s), or any combinations thereof.

Referring to FIGS. 11A and 11B, in some embodiments the first bone block 981 of the bone-tendon-bone implant 950 may be inserted into a first bone tunnel 971 that is formed in a first bone 991 (e.g., a femoral bone, etc.), and the second bone block 982 of the bone-tendon-bone implant 950 may be inserted into a second bone tunnel 972 that is formed in a second bone 992 (e.g., a tibial bone, etc.). In this manner, the soft tissue implant 985 of the bone-tendon-bone implant 950 may span a joint space (e.g., a knee joint space, etc.) intermediate the first bone block 981 and the second bone block 982.

As shown in the first circle 901 area of FIG. 11B, a first interference bone screw 900 having a first helical thread 910 and a first shaft 905 may then be inserted into the first bone tunnel 971 to couple the first bone block 981 and its associated portion of the soft tissue implant 985 within the first bone tunnel 971, as previously described herein with reference to FIGS. 9A and 9B. Likewise, a second interference bone screw 1000 having a second helical thread 1020 and a second shaft 1005 may also be inserted into the second bone tunnel 972 to couple the second bone block 982 and its associated portion of the soft tissue implant 985 within the second bone tunnel 972, as shown in the second circle 902 area of FIG. 11B.

FIGS. 12A-15B show various different interference bone screw and bone-tendon-bone implant systems that may be utilized to reconstruct a soft tissue (e.g., an ACL within a knee joint, etc.).

For example, FIGS. 12A and 12B illustrate an example system for reconstructing a soft tissue that comprises a first interference bone screw 900 including a first helical thread 910 that may be configured to couple with a first bone block 981 of a bone-tendon-bone implant 950, in order to couple the first bone block 981 and its associated portion of the soft tissue implant 985 within a first bone tunnel 971 formed in a first bone 991. Likewise, a second interference bone screw 1000 may include a second helical thread 1020 that may be configured to couple with a second bone block 982 of the bone-tendon-bone implant 950 in order to couple the second bone block 982 and its associated portion of the soft tissue implant 985 within a second bone tunnel 972 formed in a second bone 992. In this embodiment, the first helical thread 910 may comprise a standard thread orientation, and the second helical thread 1020 may comprise an inverted thread orientation. The orientation of the first helical thread 910 and the second helical thread 1020 with respect to each other may be selected for some soft tissue reconstruction scenarios that may be likely to experience certain axial, off-axis, and/or radial loading scenarios, in order to provide optimal fixation for such soft tissue reconstruction systems.

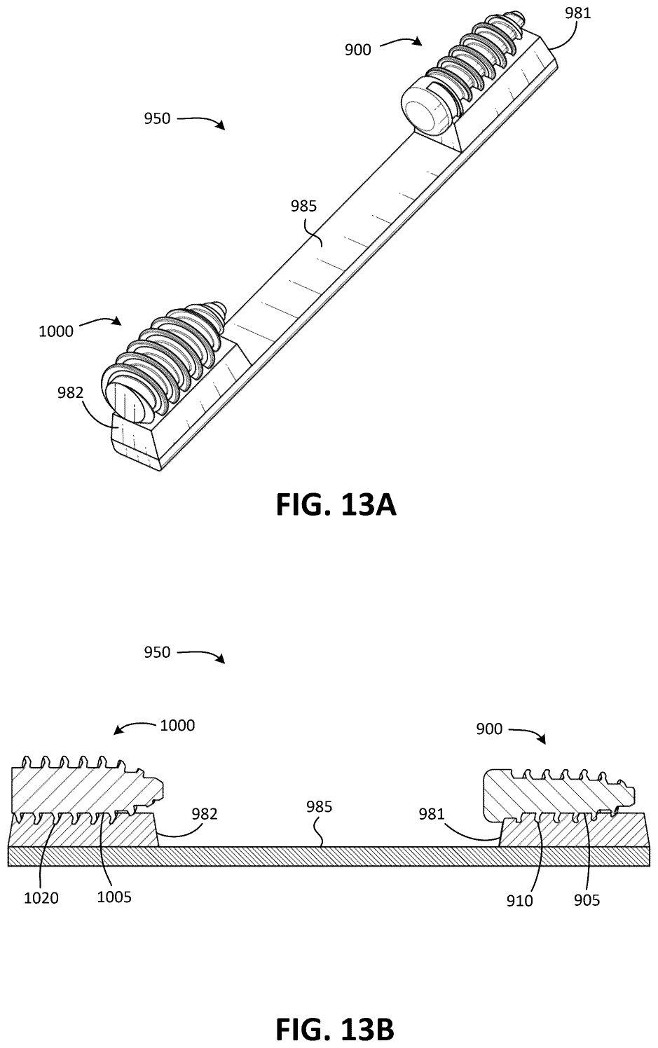

FIGS. 13A and 13B illustrate an example system for reconstructing a soft tissue that comprises a first interference bone screw 900 including a first helical thread 910 that may be configured to couple with a first bone block 981 of a bone-tendon-bone implant 950, in order to couple the first bone block 981 and its associated portion of the soft tissue implant 985 within a first bone tunnel 971 formed in a first bone 991. Likewise, a second interference bone screw 1000 may include a second helical thread 1020 that may be configured to couple with a second bone block 982 of the bone-tendon-bone implant 950, in order to couple the second bone block 982 and its associated portion of the soft tissue implant 985 within a second bone tunnel 972 formed in a second bone 992. In this embodiment, the first helical thread 910 may comprise an inverted thread orientation, and the second helical thread 1020 may comprise a standard thread orientation. The orientation of the first helical thread 910 and the second helical thread 1020 with respect to each other may be selected for some soft tissue reconstruction scenarios that may be likely to experience certain axial, off-axis, and/or radial loading scenarios, in order to provide optimal fixation for such soft tissue reconstruction systems.

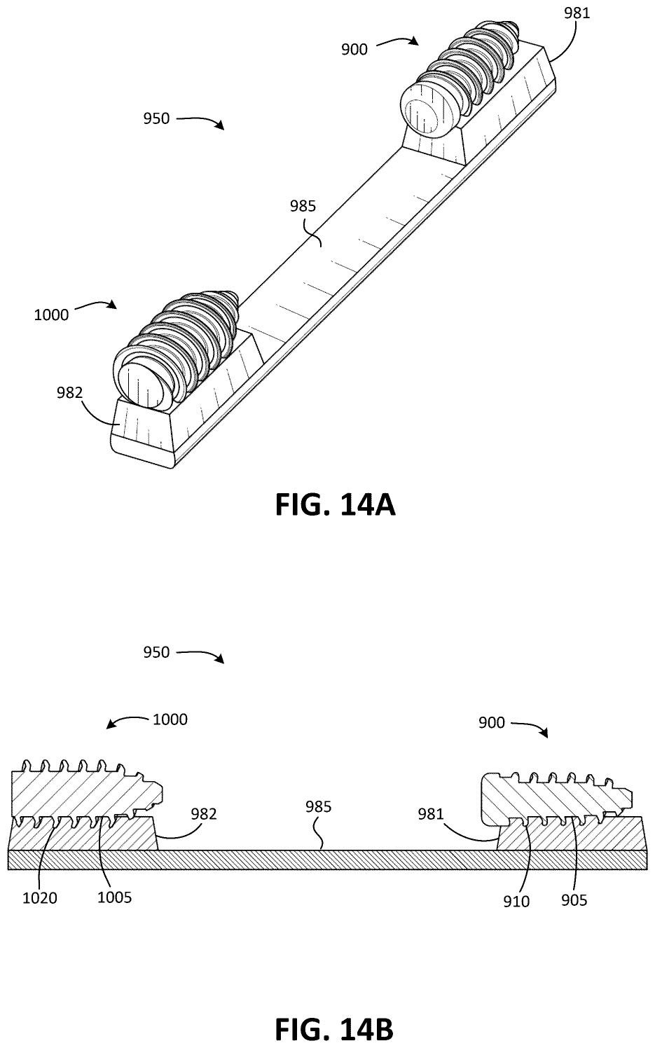

FIGS. 14A and 14B illustrate an example system for reconstructing a soft tissue that comprises a first interference bone screw 900 including a first helical thread 910 that may be configured to couple with a first bone block 981 of a bone-tendon-bone implant 950, in order to couple the first bone block 981 and its associated portion of the soft tissue implant 985 within a first bone tunnel 971 formed in a first bone 991. Likewise, a second interference bone screw 1000 may include a second helical thread 1020 that may be configured to couple with a second bone block 982 of the bone-tendon-bone implant 950, in order to couple the second bone block 982 and its associated portion of the soft tissue implant 985 within a second bone tunnel 972 formed in a second bone 992. In this embodiment, the first helical thread 910 may comprise a standard thread orientation, and the second helical thread 1020 may also comprise a standard thread orientation. The orientation of the first helical thread 910 and the second helical thread 1020 with respect to each other may be selected for some soft tissue reconstruction scenarios that may be likely to experience certain axial, off-axis, and/or radial loading scenarios, in order to provide optimal fixation for such soft tissue reconstruction systems.

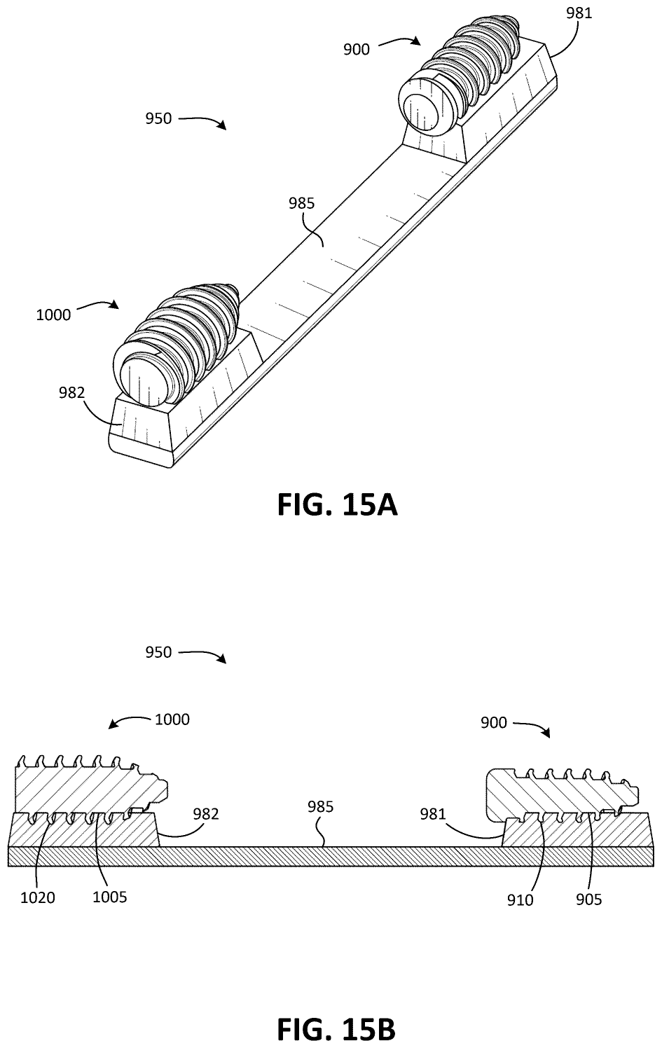

FIGS. 15A and 15B illustrate an example system for reconstructing a soft tissue that comprises a first interference bone screw 900 including a first helical thread 910 that may be configured to couple with a first bone block 981 of a bone-tendon-bone implant 950, in order to couple the first bone block 981 and its associated portion of the soft tissue implant 985 within a first bone tunnel 971 formed in a first bone 991. Likewise, a second interference bone screw 1000 may include a second helical thread 1020 that may be configured to couple with a second bone block 982 of the bone-tendon-bone implant 950, in order to couple the second bone block 982 and its associated portion of the soft tissue implant 985 within a second bone tunnel 972 formed in a second bone 992. In this embodiment, the first helical thread 910 may comprise an inverted thread orientation, and the second helical thread 1020 may also comprise an inverted thread orientation. The orientation of the first helical thread 910 and the second helical thread 1020 with respect to each other may be selected for some soft tissue reconstruction scenarios that may be likely to experience certain axial, off-axis, and/or radial loading scenarios, in order to provide optimal fixation for such soft tissue reconstruction systems.

It will be understood that any fastener, bone implant, joint replacement implant, articular member, interference bone screw, etc., that is described or contemplated herein may include any thread configuration, feature, or morphology that is described or contemplated herein, and/or may be utilized in conjunction with (or within) any system, method, or instrumentation that is described or contemplated herein.

For example, in some embodiments the first interference bone screw 900 and/or the second interference bone screw 1000 may each comprise headed fasteners, headless fasteners, or any combination of headed and headless fasteners.

In some embodiments, the lead/pitch of the thread(s) on the first interference bone screw 900 and/or the second interference bone screw 1000 may be configured to vary along at least a length of the fastener, thereby creating a cross-section of bone engagement that may also vary/change moving from a first distal position along the fastener to a second proximal position along the fastener. For example, the lead/pitch of the thread(s) may increase or decrease moving from a first distal position along the fastener to a second proximal position along the fastener, etc. In some embodiments, the lead/pitch of the thread(s) may be configured to continuously vary (and/or discretely vary) moving from a first distal position along the fastener to a second proximal position along the fastener, etc.

In some embodiments, the thickness of the thread(s) on the first interference bone screw 900 and/or the second interference bone screw 1000 may be configured vary along at least a length of the fastener, thereby tailoring the fastener thread(s) for different expected load scenarios. For example, a width of the thread may increase (i.e., become “thicker”), or decrease (i.e., become “thinner”), moving from a first distal position along the fastener to a second proximal position along the fastener, etc.

It will be understood that any fastener, bone implant, joint replacement implant, articular member, interference bone screw, etc., that is described or contemplated herein may be utilized with any other fastener, bone implant, joint replacement implant, articular member, interference bone screw, etc., that is described or contemplated herein to form a system for reconstructing a soft tissue that comprises at least two fasteners. Moreover, any of the fasteners comprising the system can separately comprise any feature, thread configuration, or morphology that is described or contemplated herein with respect to any fastener (e.g., a round head, a headless configuration with a full thread, a standard thread, an inverted thread, a single thread design, a dual thread design, a chevron shaped thread, a crescent shaped thread, etc.). In some embodiments, one or more of the fasteners in the system may be identical with each other, or substantially identical with each other, etc.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112. It will be apparent to those having skill in the art that changes may be made to the details of the above-described embodiments without departing from the underlying principles set forth herein.

The phrases “connected to”, “coupled to”, “engaged with”, and “in communication with” may refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, components that are removably and/or non-removably coupled with each other, components that are functionally coupled to each other through one or more intermediary components, etc. The term “abutting” refers to items that may be in direct physical contact with each other, although the items may not necessarily be attached together. The phrase “fluid communication” refers to two or more features that are connected such that a fluid within one feature is able to pass into another feature.

As defined herein the term “substantially” means within +/−20% of a target value, measurement, or desired characteristic.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

What is claimed is:

1. A system for reconstructing a soft tissue comprising:
a first interference bone screw comprising:
a first shaft comprising:
a first proximal end;
a first distal end; and
a first longitudinal axis; and
a first helical thread disposed about the first shaft along the first longitudinal axis between a first location and a second location along the first shaft, the first helical thread comprising a first concave undercut surface; and
a second interference bone screw comprising:
a second shaft comprising:
a second proximal end;
a second distal end; and
a second longitudinal axis; and
a second helical thread disposed about the second shaft along the second longitudinal axis between a third location and a fourth location along the second shaft, the second helical thread comprising a second concave undercut surface,
wherein:
the first helical thread of the first interference bone screw is configured to interlock with of a first bone block, the first bone block placed intermediate the first interference bone screw and a first portion of a soft tissue implant within a first bone tunnel formed in a first bone; and
the second helical thread of the second interference bone screw is configured to interlock with a second bone block, the second bone block placed intermediate the second interference bone screw and a second portion of the soft tissue implant to couple the second portion of the soft tissue implant within a second bone tunnel formed in a second bone.

2. The system of claim 1, wherein:
the first concave undercut surface of the first helical thread comprises a standard thread orientation; and
the second concave undercut surface of the second helical thread comprises an inverted thread orientation.

3. The system of claim 1, wherein:
the first concave undercut surface of the first helical thread comprises an inverted thread orientation; and
the second concave undercut surface of the second helical thread comprises a standard thread orientation.

4. The system of claim 1, wherein:
the first concave undercut surface of the first helical thread comprises a first standard thread orientation; and
the second concave undercut surface of the second helical thread comprises a second standard thread orientation.

5. The system of claim 1, wherein:
the first concave undercut surface of the first helical thread comprises a first inverted thread orientation; and
the second concave undercut surface of the second helical thread comprises a second inverted thread orientation.

6. The system of claim 1, wherein at least one of:
the first bone block comprises a first pre-formed thread configured to receive the first helical thread therein; and
the second bone block comprises a second pre-formed thread configured to receive the second helical thread therein.

7. The system of claim 1, wherein at least one of:
the first bone tunnel comprises a first bone tunnel pre-formed thread configured to receive the first helical thread therein; and
the second bone tunnel comprises a second bone tunnel pre-formed thread configured to receive the second helical thread therein.

8. A system for reconstructing a soft tissue comprising:
a first interference bone screw comprising:
a first shaft comprising:
a first proximal end;
a first distal end; and
a first longitudinal axis; and
a first helical thread disposed about the first shaft along the first longitudinal axis between a first location and a second location along the first shaft, the first helical thread comprising a first concave undercut surface; and
a second interference bone screw comprising:
a second shaft comprising:
a second proximal end;
a second distal end; and
a second longitudinal axis; and
a second helical thread disposed about the second shaft along the second longitudinal axis between a third location and a fourth location along the second shaft, the second helical thread comprising a second concave undercut surface,
wherein:
one of the first interference bone screw and the second interference bone screw is configured to interlock with a bone block engaged with a first portion of a soft tissue implant to couple the first portion of the soft tissue implant within a first bone tunnel formed in a first bone; and
the other one of the first interference bone screw and the second interference bone screw is configured to interlock with a second portion of the soft tissue implant to couple the second portion of the soft tissue implant within a second bone tunnel formed in a second bone.

9. The system of claim 8, wherein:
the first concave undercut surface comprises at least one of:
a first standard thread orientation; and
a first inverted thread orientation; and
the second concave undercut surface comprises at least one of:
a second standard thread orientation; and
a second inverted thread orientation.

10. The system of claim 8, wherein:
the first interference bone screw comprises at least one first minor diameter; and
the second interference bone screw comprises at least one second minor diameter.

11. The system of claim 10, wherein:
the at least one first minor diameter is constant along at least a portion of a first length of the first shaft; and
the at least one second minor diameter is constant along at least a portion of a second length of the second shaft.

12. The system of claim 10, wherein:
the at least one first minor diameter is configured to decrease in size toward the first distal end of the first shaft; and
the at least one second minor diameter is configured to decrease in size toward the second distal end of the second shaft.

13. The system of claim 12, wherein:
the at least one first minor diameter is configured to continuously decrease in size toward the first distal end of the first shaft; and
the at least one second minor diameter is configured to continuously decrease in size toward the second distal end of the second shaft.

14. The system of claim 12, wherein:
the at least one first minor diameter is configured to discretely decrease in size toward the first distal end of the first shaft; and
the at least one second minor diameter is configured to discretely decrease in size toward the second distal end of the second shaft.

15. A system for reconstructing a soft tissue comprising:
a first interference bone screw comprising:
a first shaft comprising:
a first proximal end;
a first distal end; and
a first longitudinal axis; and
a first helical thread disposed about the first shaft along the first longitudinal axis between a first location and a second location along the first shaft, the first helical thread comprising a first concave undercut surface; and
a second interference bone screw comprising:
a second shaft comprising:
a second proximal end;
a second distal end; and
a second longitudinal axis; and
a second helical thread disposed about the second shaft along the second longitudinal axis between a third location and a fourth location along the second shaft, the second helical thread comprising a second concave undercut surface,
wherein:
the first helical thread of the first interference bone screw is configured to interlock with a first portion of a soft tissue implant to couple the first portion of the soft tissue implant within a first bone tunnel formed in a first bone; and
the second helical thread of the second interference bone screw is configured to interlock with a second portion of the soft tissue implant to couple the second portion of the soft tissue implant within a second bone tunnel formed in a second bone.

16. The system of claim 15, wherein:
the first concave undercut surface comprises at least one of:
a first standard thread orientation; and
a first inverted thread orientation; and
the second concave undercut surface comprises at least one of:
a second standard thread orientation; and
a second inverted thread orientation.

17. The system of claim 15, wherein:
the first interference bone screw comprises at least one first major diameter; and
the second interference bone screw comprises at least one second major diameter.

18. The system of claim 17, wherein:
the at least one first major diameter is constant along at least a portion of the first helical thread; and
the at least one second major diameter is constant along at least a portion of the second helical thread.

19. The system of claim 17, wherein:
the at least one first major diameter is configured to continuously decrease in size toward the first distal end of the first shaft; and
the at least one second major diameter is configured to continuously decrease in size toward the second distal end of the second shaft.

20. The system of claim 17, wherein:
the at least one first major diameter is configured to discretely decrease in size toward the first distal end of the first shaft; and
the at least one second major diameter is configured to discretely decrease in size toward the second distal end of the second shaft.
